SEQUENCE LISTING (26 sequences)

SEQ ID NO: 1 - protein, 469 aa, Merkel cell polyomavirus
  Truncated Large T Antigen position 1 to 469
MDLVLNRKEREALCKLLEIAPNCYGNIPLMKAAFKRSCLKHHPDKGGNPVIMMELNTLWSKFQQNIHKLRSDFSMFDEVDEAPIYGTTKFKEWWRSGGFSFGKAYEYGPNPHGTNSRSRKPSSNASRGAPSGSSPPHSQSSSSGYGSFSASQASDSQSRGPDIPPEHHEEPTSSSGSSSREETTNSGRESSTPNGTSVPRNSSRTDGTWEDLFCDESLSSPEPPSSSEEPEEPPSSRSSPRQPPSSSAEEASSSQFTDEEYRSSSFTTPKTPPPFSRKRKFGGSRSSASSASSASFTSTPPKPKKNRETPVPTDFPIDLSDYLSHAVYSNKTVSCFAIYTTSDKAIELYDKIEKFKVDFKSRHACELGCILLFITLSKHRVSAIKNFCSTFCTISFLICKGVNKMPEMYNNLCKPPYKLLQENKPLLNYEFQEKEKEASCNWNLVAEFACEYELDEVRDAGNDQDRNAM

SEQ ID NO: 2 - protein, 129 aa, Pseudomonas
  N-terminal end of ExoS from position 1 to 129
MHIQSLQQSPSFAVELHQAASGRLGQIEARQVATPSEAQQLAQRQDAPKGEGLLARLGAALVRPFVAIMDWLGKLLGSHARTGPQPSQDAQPAVMSSAVVFKQMVLQQALPMTLKGLDKASELATLTPE

SEQ ID NO: 3 - protein, 129 aa, Pseudomonas
  N-terminal of ExoT from position 1 to 129
MHIQSSQQNPSFVAELSQAVAGRLGQVEARQVATPREAQQLAQRQEAPKGEGLLSRLGAALARPFVAIIEWLGKLLGSRAHASTQAPLSRQDAPPAASLSAAEIKQMMLQKALPLTLGGLGKASELATL

SEQ ID NO: 4 - protein, 13 aa, Artificial Sequence
  PADRE sequence
AKFVAAWTLKAAA

SEQ ID NO: 5 - protein, 17 aa, Influenza A virus
  NP68 peptide 362-378
GVQIASNENMDAMESST

SEQ ID NO: 6 - protein, 8 aa, Artificial Sequence
  Ovalbumine peptide 257 to 264
SIINFEKL

SEQ ID NO: 7 - DNA, 774 bp, Artificial Sequence
  nucleotide sequence encoding the amino acid sequence from position 2 to 259 of SEQ ID NO 1 which has been further mutated to position 216 (E216K), for optimized expression in Pseudomonas
gacctggttttgaatcgcaaggaacgtgaagcattgtgtaagcttctggagattgccccgaactgctacgggaatatcccgctgatgaaagccgctttcaagcgctcgtgcctgaagcaccacccggataaggggggcaacccggtcattatgatggagctgaacacgctttggtcgaagttccaacagaacatccacaagctgcgttcggacttctccatgttcgatgaggtggacgaagccccgatctacggcactaccaagttcaaagagtggtggcgctcgggcggcttctcgttcggaaaggcatacgagtatgggccgaaccctcatggcacaaacagccgttcgcgcaaaccgagtagtaacgcttcgcgcggtgccccgagcggaagctcgccgccacatagtcagagctcgtccagcgggtatggctcgtttagtgcaagccaggcaagtgattcccagagtcggggcccggatataccgcccgaacaccacgaggagcccacgtcctccagcggtagctcgagtcgggaagaaacgaccaacagcggacgcgaatcgagcacgcccaatggcacctcggttccccgcaattcctcgcgcaccgatggtacttgggaagacttgttttgcgacgagagtttgtcctccccggagcctccgagcagtagcgaggaaccggaagagccaccctcgtcccgttcgagtccgcggcaacctccgtcctcctcggcagaggaagcaagtagctcgcaattcaccgacgag

SEQ ID NO: 8 - DNA, 387 bp, Pseudomonas
  nucleotide sequence encoding the amino acid sequence of SEQ ID NO 2 (ExoS) from position 1 to 129
atgcatattcaatcgcttcagcagagtccgtctttcgccgtcgaattgcaccaggccgccagtgggcgtttgggacagattgaggcccgccaggtcgccacccccagtgaagcgcagcagttggcccagcgccaggacgcgccgaagggtgaggggctgctcgctcgcctgggcgcggcgctcgtgcgtcccttcgtggcgatcatggactggctgggcaaactgttgggctcccacgcccgcaccggcccgcagcccagtcaggacgcgcagcctgcggtcatgtcctcggccgtcgtgttcaagcagatggtgctgcagcaggcattgcccatgaccttgaagggactcgacaaggcgagcgagctggcgaccctgacaccggaa

SEQ ID NO: 9 - DNA, 39 bp, Artificial Sequence
  nucleotide sequence encoding the amino acid sequence of SEQ ID NO 4 (PADRE sequence)
gccaagttcgtcgccgcctggaccctgaaggccgccgcc

SEQ ID NO: 10 - DNA, 51 bp, Artificial Sequence
  nucleotide sequence encoding the amino acid sequence of SEQ ID NO 5 (peptide NP68) for optimized expression in Pseudomonas
ggcgtgcagatcgcctccaacgaaaatatggacgcgatggagtcgagcacc

SEQ ID NO: 11 - DNA, 27 bp, Artificial Sequence
  PCR primer
acttgggaaagttttgactggtggcaa

SEQ ID NO: 12 - DNA, 26 bp, Artificial Sequence
  PCR antisense primer
gggcctcgtcaacctagatgggaaag

SEQ ID NO: 13 - protein, 817 aa, Merkel cell polyomavirus
  Native Large T antigen from position 1 to 817
MDLVLNRKEREALCKLLEIAPNCYGNIPLMKAAFKRSCLKHHPDKGGNPVIMMELNTLWSKFQQNIHKLRSDFSMFDEVDEAPIYGTTKFKEWWRSGGFSFGKAYEYGPNPHGTNSRSRKPSSNASRGAPSGSSPPHSQSSSSGYGSFSASQASDSQSRGPDIPPEHHEEPTSSSGSSSREETTNSGRESSTPNGTSVPRNSSRTDGTWEDLFCDESLSSPEPPSSSEEPEEPPSSRSSPRQPPSSSAEEASSSQFTDEEYRSSSFTTPKTPPPFSRKRKFGGSRSSASSASSASFTSTPPKPKKNRETPVPTDFPIDLSDYLSHAVYSNKTVSCFAIYTTSDKAIELYDKIEKFKVDFKSRHACELGCILLFITLSKHRVSAIKNFCSTFCTISFLICKGVNKMPEMYNNLCKPPYKLLQENKPLLNYEFQEKEKEASCNWNLVAEFACEYELDDHFIILAHYLDFAKPFPCQKCENRSRLKPHKAHEAHHSNAKLFYESKSQKTICQQAADTVLAKRRLEMLEMTRTEMLCKKFKKHLERLRDLDTIDLLYYMGGVAWYCCLFEEFEKKLQKIIQLLTENIPKYRNIWFKGPINSGKTSFAAALIDLLEGKALNINCPSDKLPFELGCALDKFMVVFEDVKGQNSLNKDLQPGQGINNLDNLRDHLDGAVAVSLEKKHVNKKHQIFPPCIVTANDYFIPKTLIARFSYTLHFSPKANLRDSLDQNMEIRKRRILQSGTTLLLCLIWCLPDTTFKPCLQEEIKNWKQILQSEISYGKFCQMIENVEAGQDPLLNILIEEEGPEETEETQDSGTFSQ

SEQ ID NO: 14 - DNA, 2448 bp, Artificial Sequence
  nucleotide sequence encoding the amino acid sequence from position 2 to 817 of SEQ ID NO 13 for optimized expression in Pseudomonas
gacctggttttgaatcgcaaggaacgtgaagcattgtgtaagcttctggagattgccccgaactgctacgggaatatcccgctgatgaaagccgctttcaagcgctcgtgcctgaagcaccacccggataaggggggcaacccggtcattatgatggagctgaacacgctttggtcgaagttccaacagaacatccacaagctgcgttcggacttctccatgttcgatgaggtggacgaagccccgatctacggcactaccaagttcaaagagtggtggcgctcgggcggcttctcgttcggaaaggcatacgagtatgggccgaaccctcatggcacaaacagccgttcgcgcaaaccgagtagtaacgcttcgcgcggtgccccgagcggaagctcgccgccacatagtcagagctcgtccagcgggtatggctcgtttagtgcaagccaggcaagtgattcccagagtcggggcccggatataccgcccgaacaccacgaggagcccacgtcctccagcggtagctcgagtcgggaagaaacgaccaacagcggacgcgaatcgagcacgcccaatggcacctcggttccccgcaattcctcgcgcaccgatggtacttgggaagacttgttttgcgacgagagtttgtcctccccggagcctccgagcagtagcgaggaaccggaagagccaccctcgtcccgttcgagtccgcggcaacctccgtcctcctcggcagaggaagcaagtagctcgcaattcaccgacgaggagtatcgctccagcagctttaccacccctaaaaccccgccacccttcagtcgcaaacgcaagttcggtggttcgcggtccagcgccagttccgccagctccgctagctttacatccactcctccaaagcctaagaagaaccgtgaaacccccgtccccaccgacttcccgattgacctgagcgattatctgagccatgccgtttactcgaacaaaaccgtgagctgcttcgccatctacactaccagcgacaaggccatcgagttgtatgacaagatcgagaagttcaaggtggacttcaaaagccggcacgcctgcgagctcggctgcatccttctgttcatcacactgagcaaacaccgcgtatcggcgatcaaaaacttctgcagcacgttctgcacgatcagcttcctcatctgcaagggcgtgaacaagatgccggagatgtataacaacctgtgcaaaccgccgtacaagctcctgcaagagaataagccgctccttaactatgagtttcaggagaaggagaaagaggcgtcctgcaactggaacctggtggcggagtttgcgtgcgagtatgaactcgacgaccacttcattatcctcgcccactacctcgattttgctaagcctttcccgtgtcaaaagtgcgagaatcggagccgtctgaagcctcacaaagcgcatgaggcccatcattcgaacgcgaaactgttctacgaatccaagagccagaagacgatttgccaacaggcggccgacacggtccttgccaaacgtcgcctggaaatgttggagatgacacggaccgagatgctgtgcaagaagtttaagaaacacctggaacgcctccgcgacttggatactattgatttgctctactacatgggcggcgtagcgtggtactgttgcctgttcgaggagttcgagaagaagctccagaaaatcatacagctgctcacagagaacataccgaaataccgcaacatctggtttaagggcccgatcaattccggcaagaccagctttgcggcggcgctgatcgacctcttggaaggcaaggcgcttaatatcaactgtccaagcgataagctgcccttcgagctgggttgcgccctggataaattcatggtggttttcgaagatgtgaagggccagaacagcctgaataaggacctgcagcccgggcagggcattaacaatttggacaacctgcgggatcatcttgacggtgctgtggcggtcagcctcgagaaaaagcatgtcaacaagaagcatcaaatcttcccgccctgcatcgtcaccgccaatgattactttatcccgaaaaccctgatcgcccgcttctcctatacgcttcacttctccccaaaggctaatctgcgcgacagcctggaccagaatatggagattcgcaagcgccggatactgcagtcggggaccaccttgttgctgtgcctgatctggtgtctgcccgacaccaccttcaaaccatgtctgcaggaagagattaagaattggaagcagatcctccagtcggaaatcagctacggcaagttctgtcagatgattgaaaacgtcgaggccggccaggacccgctgctcaacatcctgatcgaggaagaaggacccgaagaaaccgaagagacccaggattcgggcacgtttagccag

SEQ ID NO: 15 - protein, 186 aa, Merkel cell polyomavirus
  Native small T antigen from position 1 to 186
MDLVLNRKEREALCKLLEIAPNCYGNIPLMKAAFKRSCLKHHPDKGGNPVIMMELNTLWSKFQQNIHKLRSDFSMFDEVSTKFPWEEYGTLKDYMQSGYNARFCRGPGCMLKQLRDSKCACISCKLSRQHCSLKTLKQKNCLTWGECFCYQCFILWFGFPPTWESFDWWQKTLEETDYCLLHLHLF

SEQ ID NO: 16 - DNA, 555 bp, Artificial Sequence
  nucleotide sequence encoding the amino acid sequence from position 2 to 186 of SEQ ID NO 15 for optimized expression in Pseudomonas
gacctggttttgaatcgcaaggaacgtgaagcattgtgtaagcttctggagattgccccgaactgctacgggaatatcccgctgatgaaagccgctttcaagcgctcgtgcctgaagcaccacccggataaggggggcaacccggtcattatgatggagctgaacacgctttggtcgaagttccaacagaacatccacaagctgcgttcggacttctccatgttcgatgaggtgtccaccaagtttccctgggaagagtacggcacactgaaggattacatgcaatccggatacaatgcgcggttttgccgcggccctggttgtatgctgaaacagctgcgcgacagcaaatgcgcctgcatcagttgcaaactgagccggcagcattgcagcctcaaaaccctcaagcagaagaactgcttgacctggggcgaatgcttctgttatcagtgcttcatcctctggttcggcttccccccaacttgggagagcttcgactggtggcagaagacgctcgaggagaccgactattgtttgctccatctgcatctgttt

SEQ ID NO: 17 - DNA, 318 bp, Artificial Sequence
  nucleotide sequence encoding the amino acid sequence from position 81 to 186 of SEQ ID NO 15 for optimized expression in Pseudomonas
accaagtttccctgggaagagtacggcacactgaaggattacatgcaatccggatacaatgcgcggttttgccgcggccctggttgtatgctgaaacagctgcgcgacagcaaatgcgcctgcatcagttgcaaactgagccggcagcattgcagcctcaaaaccctcaagcagaagaactgcttgacctggggcgaatgcttctgttatcagtgcttcatcctctggttcggcttccccccaacttgggagagcttcgactggtggcagaagacgctcgaggagaccgactattgtttgctccatctgcatctgttt

SEQ ID NO: 18 - DNA, 420 bp, Artificial Sequence
  nucleotide sequence encoding the amino acid sequence from position 2 to 141 of SEQ ID NO 15 for optimized expression in Pseudomonas
gacctggttttgaatcgcaaggaacgtgaagcattgtgtaagcttctggagattgccccgaactgctacgggaatatcccgctgatgaaagccgctttcaagcgctcgtgcctgaagcaccacccggataaggggggcaacccggtcattatgatggagctgaacacgctttggtcgaagttccaacagaacatccacaagctgcgttcggacttctccatgttcgatgaggtgtccaccaagtttccctgggaagagtacggcacactgaaggattacatgcaatccggatacaatgcgcggttttgccgcggccctggttgtatgctgaaacagctgcgcgacagcaaatgcgcctgcatcagttgcaaactgagccggcagcattgcagcctcaaaaccctcaagcagaagaactgc

SEQ ID NO: 19 - DNA, 183 bp, Artificial Sequence
  nucleotide sequence encoding the amino acid sequence from position 81 to 141 of SEQ ID NO 15 for optimized expression in Pseudomonas
accaagtttccctgggaagagtacggcacactgaaggattacatgcaatccggatacaatgcgcggttttgccgcggccctggttgtatgctgaaacagctgcgcgacagcaaatgcgcctgcatcagttgcaaactgagccggcagcattgcagcctcaaaaccctcaagcagaagaactgc

SEQ ID NO: 20 - DNA, 387 bp, Pseudomonas
  nucleotide sequence encoding the amino acid sequence of SEQ ID NO 3 (ExoT) from position 1 to 129
atgcatattcaatcatctcagcagaacccgtctttcgtggctgagttgagccaggccgtggccgggcgcctgggacaggtcgaggcccgccaggtggccactccccgggaggcgcaacaactggcccagcgccaggaagcaccgaagggcgagggcctgctctcccgcctgggggccgccctcgcgcgtcccttcgtggcgatcatcgagtggctgggcaaactgctggggagccgtgcccacgcctccacccaggcgccgctctcccgtcaggacgcgccgcctgccgccagtctctcggccgccgagatcaagcagatgatgctgcaaaaggcactgcccctgaccttgggcggacttggcaaggcgagcgagctggcgactttg

SEQ ID NO: 21 - DNA, 717 bp, Artificial Sequence
  nucleotide sequence encoding for the green fluorescent protein
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 22 - DNA, 642 bp, Artificial Sequence
  nucleotide sequence encoding the amino acid sequence from position 2 to 215 of SEQ ID NO 1 for optimized expression in Pseudomonas
gacctggttttgaatcgcaaggaacgtgaagcattgtgtaagcttctggagattgccccgaactgctacgggaatatcccgctgatgaaagccgctttcaagcgctcgtgcctgaagcaccacccggataaggggggcaacccggtcattatgatggagctgaacacgctttggtcgaagttccaacagaacatccacaagctgcgttcggacttctccatgttcgatgaggtggacgaagccccgatctacggcactaccaagttcaaagagtggtggcgctcgggcggcttctcgttcggaaaggcatacgagtatgggccgaaccctcatggcacaaacagccgttcgcgcaaaccgagtagtaacgcttcgcgcggtgccccgagcggaagctcgccgccacatagtcagagctcgtccagcgggtatggctcgtttagtgcaagccaggcaagtgattcccagagtcggggcccggatataccgcccgaacaccacgaggagcccacgtcctccagcggtagctcgagtcgggaagaaacgaccaacagcggacgcgaatcgagcacgcccaatggcacctcggttccccgcaattcctcgcgcaccgatggtacttgggaagacttgttttgcgac

SEQ ID NO: 23 - DNA, 774 bp, Artificial Sequence
  nucleotide sequence encoding the amino acid sequence from position 1 to 259 of SEQ ID NO 1 which has been further mutated to position 216 (E216K)
atggaccttgtccttaacaggaaggagcgtgaggctttgtgcaagctgctggaaatcgctccaaactgctacggtaacatccctctgatgaaggctgccttcaagcgaagctgtctcaagcaccatcctgacaagggtggaaaccctgtgatcatgatggagctcaacaccctgtggtcaaaattccagcaaaacattcacaagctccgatccgacttctccatgtttgacgaggttgacgaagcccctatatacgggaccaccaagtttaaagagtggtggaggtccggaggattcagctttgggaaagcatacgagtatgggcccaacccccacggaaccaattcccggtcccggaaaccttcctccaatgcttccaggggcgcaccaagcggttcttcccctccacattctcaatcttccagctccggttatggctctttctccgcatcacaggcctcagattctcagagcagaggcccagatattcctccagaacatcacgaggagccaacttctagtagcgggtcaagtagtagagaggagactaccaatagcggcagagagagcagcacaccaaatggcacaagcgtacctcgcaatagcagccgcacagatggcacatgggaagatttgttttgtgacaaatcactgtcttctcctgagccccccagttcttctgaggaacccgaagaacccccatcatcacgcagtagtccccggcagcccccctcttcaagtgccgaagaagccagtagttctcagtttactgat

SEQ ID NO: 24 - DNA, 564 bp, Foot-and-mouth disease virus
  Nucleotide sequence encoding an IRES
ccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataat

SEQ ID NO: 25 - DNA, 235 bp, Artificial Sequence
  nucleotide sequence of promotor EF1a
cgtgaggctccgatgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacaca

SEQ ID NO: 26 - protein, 928 aa, Homo sapiens
  amino acid sequence of pRb
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

(19) United States
(12) Patent Application Publication
(10) Pub. No.: US 2018/0201940 A1
(43) Pub. Date: Jul. 19, 2018

(54) THERAPEUTIC VACCINE FOR TREATING OR PREVENTING MERKEL CELL POLYOMA VIRUS-ASSOCIATED TUMORS

(71) Applicants: APCURE SAS, LYON (FR); ALTRABIO, LYON (FR)

(72) Inventors: Laurent BUFFAT, LYON (FR); Roberto PIERINI, LYON (FR)

(21) Appl. No.: 15/579,233

(22) PCT Filed: Jun. 2, 2016
(86) PCT No.: PCT/EP2016/062538

§ 371 Date: Dec. 4, 2017

(30) Foreign Application Priority Data
Jun. 5, 2015 (EP) 15305861.5

(51) Int. Cl. C12N 15/62; A61K 39/00; A61K 39/12; C07K 14/025; A61P 35/00

(52) U.S. Cl. CPC C12N 15/625; A61K 39/0011; A61K 2039/5252; C07K 14/025; A61P 35/00; A61K 39/12

(57) ABSTRACT

The invention relates to a therapeutic vaccine useful against tumors exhibiting as signature the Large T antigen (LT) of the Merkel Cell PolyomaVirus (MCPyV). Additionally, the therapeutic vaccine may be also useful for preventing tumors in healthy individuals infected with MCPyV. The therapeutic vaccine involves a type-3 secretion system (T3SS) bacterial vector able to deliver a polypeptide comprising LT epitopes to antigen presenting cells (APCs), such as a truncated form of LT. The invention also relates to a fusion protein comprising a truncated form of LT.

The invention relates to a therapeutic vaccine useful against tumors exhibiting as signature a truncated form of the Large T antigen (LT) of the Merkel Cell Polyoma Virus (MCPyV). Additionally, the therapeutic vaccine may also be useful for preventing tumors in healthy individuals infected with MCPyV. The therapeutic vaccine involves a type-3 secretion system (T3SS) bacterial vector able to deliver a polypeptide comprising MCPyV LT epitopes to antigen presenting cells (APCs).

In a general manner, anti-tumor responses generated by individuals are T-cell-mediated. More specifically, tumor-specific CD8 cytotoxic T-cells are involved. However, T-cells cannot recognize and therefore cannot respond to free antigen. T-cells can only “see” an antigen that has been processed and presented by cells via carrier molecules called Major Histocompatibility Complex (MHC) molecules. Naïve T-cells (i.e., T-cells that have not been exposed to antigen) can be primed/activated by a specific class of cells designated under the term “Antigen-Presenting Cells” (APCs) among which dendritic cells (DCs) are the most important, with the broadest range of antigen presentation. APCs express MHC class II as well as class I molecules and can therefore stimulate CD4 and CD8 T-cells, respectively.

Over the past 40 years, bacterial vectors for in vivo delivery of heterologous antigens into APCs have been developed. Bacterial vectors offer multiple advantages: (1) there are several well-characterized virulence-attenuation mutations; (2) the number, the amount and the in vivo location of antigen expression can be regulated; (3) multiple vaccine delivery routes are possible; and (4) they potently stimulate the innate and adaptive immune systems.

Bacteria are involved in a very wide diversity of biotic associations, ranging from biofilms to mutualistic or pathogenic associations with larger host organisms. Protein secretion plays a central role in modulating all of these interactions. In Gram-negative bacteria, six secretion pathways have been reported until now, each of them being essentially specialized in the translocation of a specific range of proteins. One of them, the type-3 secretion system (T3SS), has been more particularly explored to achieve the delivery of antigenic proteins into APCs, as it can deliver proteins across the bacterial and host cell membranes, into the cytosol of host cells.

The structural core element of T3SS is a syringe-like needle complex (also known as injectisome and involving about 30 proteins), which is embedded across the inner and outer bacterial membranes and extends into the extracellular space. In more details, this syringe-like needle complex comprises two essential components: (i) a membrane-anchored base ring and (ii) an extracellular needle (Tseng et al, BMC Biotech. (2009) 9 (suppl 1): 52). When expressed by the bacteria, T3SS may exist in an active or inactive state, depending on a number of natural switch factors. T3SS is a critical virulence factor used by several animal and plant pathogens, to deliver into the cytosol of host cells i.e., a number of exotoxins generically referred to as T3SS exotoxins. T3SS exotoxins are addressed to T3SS thanks to a short signaling sequence. When fusing an antigen of interest to this signaling sequence, the recombinant protein can be engaged in T3SS and delivered to the cytosol of epithelial cells and circulating DCs, upon appropriate administration of the bacterial vector.

Merkel Cell Polyoma Virus (MCPyV) has first been identified in 2008 as responsible for the oncogenesis of Merkel Cell Carcinoma (MCC) (Shuda et al, 2008; Feng et al, 2008), a rare but devastating disease of the skin, and is now detected in all MCC cases (Rodig et al, 2012). Cells are primarily infected with MCPyV, the viral genome of which may remain in a non-integrative form for an indefinite period of time. MCC arises from a two-step process, in which the viral genome (i) integrates into the host genome at any stage of infection and (ii) develops LT truncation mutations to prevent autonomous viral genome replication. Failure to truncate LT may lead to DNA damages responses or immune recognition that hinders nascent tumor cell survival.

More recently, the presence of MCPyV has been detected in at least 20% of non-small cell lung cancer (NSCLC) patients (Hashida et al, 2013) and a role of MCPyV in the NSCLC oncogenesis has been suggested (Antoniou et al, 2013). Also, there is an increasing evidence of MCPyV detection in several types of cancer: cutaneous squamous cell carcinoma (Murakami et al, 2011; Dworkin et al, 2009; Kassem et al, 2009; Scola et al, 2012; Karia et al, 2013), chronic lymphocytic leukemia (Cimino et al, 2013; Pantulu et al, 2010), cutaneous B- and T-cell lymphoma (Kreuter et al, 2011; Duthanh et al, 2013); and cervical squamous cell carcinoma and adenocarcinoma (Imajoh et al, 2012).

As already mentioned above, expression of truncated forms of the large T viral antigen (LT) is the signature of MCPyV-associated tumors (Shuda et al, PNAS (2008) 105 (42): 16272). Indeed, LT is a protein of about 817 amino acids and has on the host cell, both growth-promoting and inhibitory activities (Cheng et al, J. Virol. (2013) 87 (11): 6118). The N-terminal domain, including the LXCXE motif of the human retinoblastoma protein binding site (RBS), is responsible for growth-promoting activity, while the C-terminal domain (last 100 amino acids) of the protein is responsible for growth-inhibiting activity. Upon truncation mutation, the C-terminal domain may be lost, the N-terminal domain still being expressed.

As already mentioned above, expression of LT truncated forms first requires integration into the cell genome of a viral nucleotide sequence encoding LT, at a location accessible for transcription. Should a mutation event occur, introducing a stop codon downstream the region encoding i.e., the RBS (exon 2) and upstream of the region encoding the C-terminal domain (exon 3), then oncogenic LT truncated forms may be expressed.

The oncogenic effect primarily operates upon binding of the LT truncated form to the human retinoblastoma protein (pRb) which is a cellular protein regularly involved in the negative regulation of cell growth. LT-binding inactivates pRb which is therefore unable to suppress tumor growth events.

Zeng et al, Vaccine (2012) 30 (7): 1322 have shown that in a mouse model, a prophylactic DNA vaccine encoding a truncated form of the large T antigen of the MCPyV generates anti-tumor effects mainly mediated by LT-specific CD4 helper T-cells. This cannot however be considered as a fully promising result toward the making of a vaccine for MCPyV-positive tumors, since there is a general consensus for an effective anti-tumor vaccine to be able to induce a strong, and otherwise predominant CD8+ immune response (versus CD4+) against tumoral cells. Indeed, the importance of CD8 T-cells in the control and eradication of viruses has been acknowledged for long. Furthermore, tumor-reactive CD8 cytotoxic T-cells have consistently been found with improved patient outcomes.

In view of this, Gomez et al, Cell & Bioscience (2012) 2: 36 have attempted to improve the DNA vaccine of Zeng et al, so that the CD4 T-cell response be switched to a predominant CD8 T-cell response. To this end, they have generated a DNA vaccine encoding a truncated LT antigen tagged to calreticulin (CRT). CRT is a chaperone protein naturally located in the endoplasmic reticulum (ER) where it may participate to correct folding of newly made proteins. Calreticulin has also been shown to associate with peptides delivered into the ER by transporters associated with antigen processing (TAP-1 and TAP-2) and with MHC class I-β2 microglobulin molecules and has further demonstrated the ability to enhance peptide presentation to MHC class I molecules, therefore promoting the induction of antigen-specific CD8 T-cells (Basu et al, J. Exp. Med. (1999) 189 (5): 797). Consistently, Gomez et al have shown that in a mouse tumor model, a therapeutic DNA vaccine encoding truncated LT tagged to CRT improved mice survival, compared to a DNA vaccine encoding truncated LT alone, which did not perform any better than the negative control (FIG. 5 of Gomez et al).

CRT ability to enhance antigen-specific immune response can only be exploited in the context of vaccine technologies based on nucleic acid transfer and subsequent protein neosynthesis. Indeed, neosynthetized proteins enter the ER before degradation into peptides, while this is not the case when proteins are directly delivered to the cells. Indeed, for antigen presentation in the ER, ready-made proteins are first degraded into peptides upon translocation from the cytoplasm into ER. Accordingly, the use of a CRT tag is irrelevant in the context of vaccine technologies based on protein transfer (no DNA or RNA vaccine).

Surprisingly, against the teaching of Gomez et al, it has now been found that an LT-specific CD8 T-cell response can be generated, while delivering LT via a vaccine technology based on protein transfer which accordingly does not involve the use of a CRT tag. More specifically, a bacterial vector able to express LT (in the absence of CRT tag) and to secrete and transfer, preferably translocate, LT into mammalian cells via a type-3 secretion system (T3SS), was used.

In addition to this, despite the fact that bacterial vectors owning a T35S are proposed in the art for antigen delivery, not all proteins have the potential to move into T3SS, although fused to appropriate T3SS signaling sequence. This is shown in particular in Radics et al, 2014 which reports that fusion constructs wherein GFP (Green Fluorescent Protein) is fused at the C-termini of otherwise-allowed T3SS substrates, are expressed but not secreted. Indeed, their structure might be too rigid to get unfold for translocation through the T3SS needle. As the tridimensional structure of LT still remains unknown, there is no evidence in the art that LT of MCPyV could be a molecule flexible enough to be efficiently secreted via T3SS.

This is the reason why the invention relates to a bacterial vector owning a type 3 secretion system (T3SS bacterial vector), said bacterial vector being able to express, secrete and transfer, preferably translocate, into mammalian cells, a fusion protein which comprises from its N-terminal end to its C-terminal end and fused in frame:
at least one secretion peptide signal that directs said fusion protein to the type 3 secretion system of the bacterial vector, and
one truncated form of the Large T antigen (LT) of the Merkel Cell Polyoma Virus (MCPyV), wherein the LT truncated form has an amino acid sequence having at least 80% identity with one of the amino acid sequences shown in SEQ ID NO: 1 which start with the amino acid in any one of positions 1 to 5 and end with the amino acid in any one of positions 210 to 469.

In a preferred embodiment, said fusion protein does not bind to the human retinoblastoma protein.

The invention also relates to a fusion protein which comprises from its N-terminal end to its C-terminal end and fused in frame:
at least one secretion peptide signal able to direct said fusion protein to the type 3 secretion system of a bacterial vector, when said fusion protein is in a bacterial vector owning a type 3 secretion system, and
one truncated form of the Large T (LT) antigen of the Merkel Cell Polyoma Virus (MCPyV), which has an amino acid sequence having at least 80% identity with one of the amino acid sequences shown in SEQ ID NO: 1 which start with the amino acid in any one of positions 1 to 5 and end with the amino acid in any one of positions 210 to 469.

In a preferred embodiment, said fusion protein does not bind to the human retinoblastoma protein.

Another object of the invention relates to a bacterial vector of the invention for use in a method of combating MCPyV infection, in particular by promoting a CD8+ immune response against MCPyV-infected cells.

Another object of the invention relates to the bacterial vector of the invention for use as a medicament, preferably for use as a medicament in the treatment of cancer.

The present invention will first be described in general overview. Then, each element will be described in further detail below.

In the following description the standard one letter nucleic acid and the standard one letter amino acid code is used. The standard three letters amino acid code may also be used. All protein descriptions are made from its N-terminal domain to its C-terminal domain.

In practicing the present invention, many conventional techniques in molecular biology and recombinant DNA technology are used. Such techniques are well-known and are explained fully in, for example, Sambrook et al., 2012, Molecular Cloning: A Laboratory Manual, Fourth Edition, Cold Spring Harbor Laboratory Press.

By “vector owning a type 3 secretion system (T3SS vector)” is meant a vehicle having a functional type 3 secretion system capable of delivering proteins from inside of the vehicle into mammalian cells directly or indirectly. In particular, the vector is able to secrete and transfer, preferably translocate, proteins into mammalian cells. More preferably, the vector is able to express, secrete and transfer, preferably translocate, proteins into mammalian cells.

According to the invention, the vector is preferably a vector of bacterial nature or bacterial origin and is called a bacterial vector.

A bacterial vector of the invention may be a vector of bacterial origin such as particles derived from replication-deficient bacteria, such as bacterial minicells, or may be a vector of bacterial nature such as a bacterium, and in particular a modified bacterium.

Bacterial minicells are small, semi-spherical, bacterial nano-particles that contain all of the components of the parental bacteria, except chromosomes. Without chromosomes, they cannot divide and are non-infectious. Minicells may comprise the type 3 secretion system, thereby allowing the minicells to efficiently deliver proteins into mammalian cells. Bacterial minicells are described by H. A Carleton et al. Nature Communications 4, Article number: 1590 doi:10.1038/ncomms2594.

Preferably, the bacterial vector is a genetically modified bacterium and in particular an attenuated bacterium.

Advantageously, the bacterium vector is a bacterium which is less toxicogenic than the wild-type corresponding bacterium. Advantageously, toxicogenicity can be reduced to an extent that is sufficient to label a bacterial vector as non-toxicogenic. Typically, a bacterial vector is a bacterium that is acknowledged (before clinical use), as being suitable for administration to human beings, and especially to cancer patients.

In one preferred embodiment, the T3SS bacterial vector owning a type 3 secretion system may be a bacterium belonging to any bacterium genus owning a T3SS, for instance to the genus of Gram-negative bacteria. Gram-negative bacteria owning a T3SS are for example Salmonella, Shigella, Yersinia and Pseudomonas. In a preferred embodiment, the bacterial vector is a bacterium which belongs to the genus of Pseudomonas, in particular to the bacterial species Pseudomonas aeruginosa or Pseudomonas syringae.

A T3SS bacterial vector according to the invention is typically a T3SS bacterium which derives from a wild-type T3SS bacterium and is significantly less toxicogenic than the corresponding wild-type bacterium. In one embodiment, the bacterial vector according to the invention is unable to express at least one of the products chosen among the exoS, exoT, exoU and exoY gene products and NDK cytotoxin, preferably is unable to express at least the exoS, exoT and exoU gene products. Advantageously, a T3SS bacterial vector is unable to express at least one, advantageously at least two or three T3SS exotoxins of the wild-type bacterium. Toxicogenicity may be reduced by inactivating at least one T3SS exotoxin genes (T3SS exo genes). Inactivation may be achieved by mutation i.e., conveniently by deletion of whole or part of the gene. In a further improvement, exo genes other than T3SS exo genes may also be inactivated.

In a particular embodiment of the invention, the bacterial vector is a bacterium belonging to the Pseudomonas genus with reduced toxicogenicity in that it is unable to express and/or secrete at least one, advantageously two T3SS exotoxins including i.e., ExoS, ExoT, ExoU, ExoY and NDK (nucleoside diphosphate kinase) cytotoxin; and preferably at least the ExoS, ExoT and ExoU exotoxins. Inability to express and/or secrete an exo gene product can be achieved by inactivating the exo gene, e.g., by deleting whole or part of the exo gene. A Ps. aeruginosa bacterium unable to express at least one exotoxin (ExoU) is the CHA strain, available in the art (Toussaint et al, Biochem. Biophys. Res. Commun. (1993) 196: 416). A Ps. aeruginosa bacterium unable to express the ExoS, ExoT and ExoU exotoxins is the CHA strain exhibiting additional ΔexoS and ΔexoT inactivations; such a strain is available in the art under the name CHA-OST or CHAΔSTlox (Quénée et al, BioTechniques (2005) 38: 63). The CHA strain may also be available at the Collection Nationale de Culture de Microorganismes (CNCM, Institut Pasteur, Paris) under the name CHA-003 and reference number 1-3090 (deposited on Sep. 17, 2003, under the Budapest Treaty by the University Joseph Fourier (Grenoble, FR). For more details, it is possible to refer to WO 2005/049644.

In the following description, the expression “bacterial vector” or “T3SS bacterial vector” “T3SS vector” are synonymous.

By “express”, it is meant that the bacterial vector is able to translate nucleic sequences, thereby allowing the synthesis of proteins encoded by said nucleic sequences. The protein expression may be checked by any one of the biochemistry technologies well-known in the art, such as Western Blot analysis of lysate of the bacterial vector. The protein expression may also be checked as described in Epaulard O et al, Mol. Ther. (2006) 14: 656.

By “secrete and transfer into mammalian cells”, it is meant that the bacterial vector is able to send proteins having a secretion peptide signal at distance into mammalian cells, more specifically into the cytosol of the mammalian cells. Therefore, said proteins are displaced from a place to another place, said place being separated by membranes. The delivery occurs preferably directly from the bacterial vector into the mammalian cell. In this case, the secretion and the transfer correspond to a translocation. It is also possible that the protein is intermediary delivered in the environment surrounding the bacterial vector and the mammalian cell and after transferred in the mammalian cell (Edgren T et al. PloS Pathog. 2012; 8(5):e1002669. doi: 10.1371/journal.ppat.1002669. Epub 2012 May 10.). The secretion peptide signal is recognized by the complex of the T3SS system. These proteins are then transported via the T3SS system from the interior of the bacterial vector (such as from the cytosol of the bacterial vector), to its exterior and are preferably injected directly into the cytoplasm of the mammalian cells. The T3SS acting in this case like a syringe. Proteins pass through the inner membrane, the periplasmic space and the outer membrane of the bacterial vector, and the membrane of the mammalian cells. The delivery, i.e the secretion and transfer, of proteins can be assessed by the methods described in the example. The secretion of protein may be assessed by a secretion assay. This consists in the detection of the secreted protein in the bacterial supernatant. The detection is done on an acrylamide gel and comassie staining or by Western Blot. An example of a secretion assay is described in Epaulard O et al, Mol. Ther. (2006) 14: 656.

By “translocate into mammalian cells,” it is meant that the secretion and the transfer of proteins into the mammalian cells, such as into the cytosol of the mammalian cells, occur directly, the proteins being directly displace from the bacterial vector to the mammalian cell, through the T3SS system, acting like a syringe. The translocation can be assessed by the methods described in the example or in Le Gouëllec A et al Mol Ther. 2013 May; 21(5):1076-86. doi: 10.1038/mt.2013.41. Epub 2013 Mar. 26. PMID: 23531551.

By “mammalian cells”, it is meant, any animal, preferably human, eukaryotic cells present in vitro, ex vivo or in vivo in research, diagnostic or therapeutic applications. For example, mammalian cells may be cells of the immune system, in particular antigen presenting cells (APCs) like monocytes, dendritic cells, macrophages, B cells and NK cells. In one preferred embodiment, the mammalian cells are dendritic cells.

By “fusion protein”, it is meant a protein artificially created from at least two peptides fragments (or moieties) of different origins, which are fused either directly (generally by a peptide bond) or via a peptide linker. The two peptides fragments are encoded by amino-acid sequences of different origins. In particular, the fusion protein comprises at least one fragments of bacterial origin and one fragment of virus origin. According to the invention, the fragment of bacterial origin is related to a secretion peptide signal that directs said fusion protein to the T3SS system of a bacterial vector and the fragment of the virus origin is related to a truncated form of the Large T antigen of the Merkel Cell Polyoma virus.

Fusion of the various moieties composing the fusion protein can classically be achieved by a direct peptide bond or by small peptides, advantageously made of at most 1, 2, 3 or 4 amino acids.

The at least two sequences are fused using techniques known to those skilled in the art and, more precisely described in Sambrook et al., 2012, Molecular Cloning: A Laboratory Manual, Fourth Edition, Cold Spring Harbor Laboratory Press.

By “truncated form of the Large T antigen (LT)” it is meant a LT antigen that is shortened compared to the native form of the LT antigen. Therefore, the truncated form of the LT antigen is a fragment of the LT antigen and has a length of less than 817 amino acids.

More specifically, the truncated form of the Large T antigen used in the fusion protein may have an amino acid sequence having at least 80% identity with one of the amino acid sequences shown in SEQ ID NO:1 which starts with an amino acid in any one of the positions 1 to 5, advantageously in position 2, and ends with an amino acid in any one of the positions 210 to 469, and, preferably, provided that said truncated form of the Large T antigen used in the context of the invention does not bind to the human retinoblastoma protein.

The following expression “the LT truncated form Z has an amino acid sequence shown in SEQ ID NO: Y which starts with the amino acid in any one of positions i to j (i and j are integers and j>i) and ends with the amino acid in any one of positions n to m (n and m are integers and m>n)” means that the LT truncated form Z has any one of the amino acid sequences consisting of the amino acid sequence of SEQ ID NO: Y which starts with any one of the amino acid in positions i to j of the SEQ ID NO: Y and ends with any one of the amino acid in positions n to m of the SEQ ID NO: Y. In particular, the expression encompasses a LT truncated form Z encoded by the SEQ ID NO: Y (i->n) (also called LT(i-n)), a LT truncated form Z encoded by the SEQ ID NO: Y (i->m) (also called LT(i-m)), a LT truncated form Z encoded by the SEQ ID NO: Y (j->n) (also called LT(j-n)) and a LT truncated form Z encoded by the SEQ ID NO: Y (j->m) (also called LT(j-m)).

When it is said that a LT truncated form of the LT antigen of MCPyV has an amino acid sequence, it means that the amino acid sequence of the LT truncated form comprises at least and at most the given defined amino acid sequence.

By “not bind to the human retinoblastoma protein”, it is meant that the fusion protein, and more particularly the truncated form of LT, is not able to be associated with the human retinoblastoma protein (pRB) by any attractive interaction, such as non-covalent liaison, in particular hydrogen bond, ionic interactions, Van der Waals forces or hydrophobic bonds; therefore pRB is still able to suppress tumour growth events. The fusion protein is not able to bind to the pRB because the truncated form of the LT protein has either a non-functional human retinoblastoma protein binding site (RBS) or no human retinoblastoma protein binding site at all. The human retinoblastoma protein (abbreviated pRb), is a tumor suppressor protein that is dysfunctional in several major cancers. One function of pRb is to prevent excessive cell growth by inhibiting cell cycle progression until a cell is ready to divide. By “human retinoblastoma protein” it si meant a protein encoded by the RB1 gene (also named RB gene) located on 13q14.1-q14.2 of the human genome and in particular the “human retinoblastoma protein” designates the protein of SEQ ID NO: 26.

In one embodiment, a non-functional human retinoblastoma protein binding site (RBS) may be obtained by introducing mutation in the RBS of the truncated LT antigen when said truncated LT antigen contains at least the first 216 amino acids of the native form of the LT antigen. The RBS located from positions 212 to 216 in SEQ ID NO: 1 is mutated so that it is rendered non-functional, leading to an LT truncated form and a fusion protein unable to bind pRb. While the RBS may be mutated in any one of positions 212 to 216, it is preferred to mutate in position 212, 214 or 216. As a matter of example, a useful mutation is a mutation substituting the glutamic acid in position 216 by another amino acid which is not glutamine, acid aspartic or asparagine. Advantageously, the substitution amino acid is a neutral or positively-charged amino acid. A useful neutral amino acid may be alanine, valine, leucine, isoleucine, methionine, phenylalanine, threonine or tryptophan. A useful positively-charged amino acid may be arginine, histidine or lysine, this latter being preferred.

As a matter of example, a useful mutation is a mutation substituting the glutamic acid residue in position 216 by a lysine residue. An example of truncated form of LT having a non-functional RBS is a truncated form of LT having an amino acid sequence shown in SEQ ID NO: 1 which starts with an amino acid in any one of the positions 1 to 5, advantageously in position 2, and ends with the amino acid in any one of the positions 210 to 469 and is further mutated in the human retinoblastoma protein binding site located from positions 212 to 216 in SEQ ID NO: 1. Accordingly, a typical example of an LT truncated form having a non-function RBS (i.e. a LT truncated and mutated form) is LT(2-259, E216→K) by reference to SEQ ID NO: 1. In another embodiment, an LT truncated form having a non-functional RBS may be obtained by substituting any one of the amino acids located from positions 212 to 216 in SEQ ID NO: 1 by a codon stop. Accordingly, a typical example of an LT truncated form having a non-functional RBS is LT(2-215).

Mutation in SEQ ID NO: 1 may be obtained by molecular techniques well-known in the art and more precisely described in Sambrook et al., 2012, Molecular Cloning: A Laboratory Manual, Fourth Edition, Cold Spring Harbor Laboratory Press.

The ability of the fusion protein to bind or not to the pRB (and the ability to test if a truncated LT form has a non-functional RBS) may be studied by techniques well-known by the skilled person in the art such as surface plasmon resonance or immunoprecipitation as described in Shuda et al, Proc. Nat. Acad. Sci (2008), 105 (42):16272.

In a particular embodiment, the truncated form of the LT antigen of the MCPyV of the fusion protein has:
i. an amino acid sequence having at least 80% identity with the amino acid sequence shown on SEQ ID NO: 1 which starts with the amino acid in position 2 and ends with the amino acid in position 215, and, preferably, provided that said truncated form of the LT antigen of the MCPyV does not bind to the human retinoblastoma protein, or
ii. an amino acid sequence having at least 80% identity with the amino acid sequence shown on SEQ ID NO: 1 which starts with the amino acid in position 2 and ends with the amino acid in position 270, and, preferably, provided that said truncated form of the LT antigen of the MCPyV does not bind to the human retinoblastoma protein, or
iii. one of the amino acid sequences shown in SEQ ID NO: 1 which starts with the amino acid in any one of positions 1 to 5, advantageously in position 2, and ends with the amino acid in any one of positions 210 to 215, so that said truncated form of the LT antigen of the MCPyV does not bind to the human retinoblastoma protein; or
iv. one of the amino acid sequences shown in SEQ ID NO: 1 which starts with the amino acid in any one of positions 1 to 5, advantageously in position 2, and ends with the amino acid in any one of positions 216 to 270, which is further mutated in the human retinoblastoma protein binding site located from positions 212 to 216 in SEQ ID NO: 1, so that said truncated form of the LT antigen of the MCPyV does not bind to the human retinoblastoma protein.

In a particular embodiment, the amino acid sequence of the LT truncated form of the fusion protein expressed, secreted and transferred, preferably translocated by the T3SS bacterial vector according to the invention has at least 80%, 85%, 90%, 95%, or advantageously 100% identity, with the amino acid sequence shown in SEQ ID NO: 1 which starts with the amino acid in position 2, and ends with the amino acid in position 469 and, preferably, provided that said LT truncated form is not able to bind the pRB.

In a particular embodiment, the amino acid sequence of the LT truncated form of the fusion protein expressed, secreted and transferred, preferably translocated by the T3SS bacterial vector according to the invention has an amino acid sequence shown in SEQ ID NO: 1 which starts with the amino acid in any one of positions 1 to 5, advantageously in position 2, and ends with the amino acid in position 215.

In a particular embodiment, the amino acid sequence of the LT truncated form of the fusion protein expressed, secreted and transferred, preferably translocated by the T3SS bacterial vector according to the invention has:
a one of the amino acid sequences shown in SEQ ID NO: 1 which starts with the amino acid in any one of positions 1 to 5, advantageously in position 2, and ends with the amino acid in any one of positions 250 to 260, advantageously in position 259, which is further mutated in the human retinoblastoma protein binding site located from positions 212 to 216 in SEQ ID NO: 1, so that said LT truncated form does not bind to the human retinoblastoma protein; or
an amino acid sequence having at least 85% identity with the amino acid sequence shown in SEQ ID NO: 1 which starts with the amino acid in position 2 and ends with the amino acid in position 259; and, preferably, provided that LT truncated form does not bind to the human retinoblastoma protein.

In another particular embodiment, the amino acid sequence of the LT truncated form of the fusion protein expressed, secreted and transferred, preferably translocated by the T3SS bacterial vector according to the invention has at least 80%, 85%, 90%, 95%, or advantageously 100% identity, with the amino acid sequence shown in SEQ ID NO: 1 which starts with the amino acid in position 2, and ends with the amino acid in position 215 and, preferably, provided that LT truncated form does not bind to the human retinoblastoma protein. A typical example of this is LT(2-215) by reference to SEQ ID NO: 1.

In another particular embodiment, the LT truncated form of the fusion protein expressed, secreted and transferred, preferably translocated by the T3SS bacterial vector according to the invention is defined by an amino acid sequence consisting of an amino acid sequence shown in SEQ ID NO: 1 which starts with the amino acid in any one of positions 1 to 5, advantageously in position 2, and ends with the amino acid in any one of positions 216, 220, 230, 240 or 250 to 270, advantageously in any one of positions 250 to 260, preferably in position 259; which is further mutated in the human retinoblastoma protein binding site (RBS) located from position 212 to 216 in SEQ ID NO: 1, so that the fusion protein is unable to bind the human retinoblastoma protein (pRb) and in particular so that said LT truncated form has a non-functional RBS.

For the sake of brevity, the term “LT truncated forms” will herein after includes all the LT truncated forms within the scope of the invention, including the LT truncated and mutated forms and the LT truncated form having no RBS site such as LT truncated forms having an amino acid sequence consisting of the amino acid sequence shown in SEQ ID NO: 1 which starts with the amino acid in any one of positions 1 to 5, advantageously in position 2, and ends with the amino acid in any one of position 210 or 211.

Alternatively, the amino acid sequence of the LT truncated form of the fusion protein expressed, secreted and transferred, preferably translocated, by the T3SS bacterial vector according to the invention has:
at least 80%, 85%, 90% or 95% identity, with the amino acid sequence shown in SEQ ID NO: 1 which starts with the amino acid in position 2 and ends with the amino acid in position 270; or
at least 85%, 90%, 95%, 97% or 99% identity with the amino acid sequence shown in SEQ ID NO: 1 which starts with the amino acid in position 2 and ends with the amino acid in position 259;
   and, preferably, provided that said LT truncated form is unable to bind the human retinoblastoma protein (pRb), in particular provided that LT truncated form has a non-functional RBS site.

In yet another embodiment, LT truncated form contained in the fusion protein according to the invention is the sole antigen comprising MCPyV epitopes able to be expressed, secreted and transferred, preferably translocated by the bacterial vector according to the invention. That means that the fusion protein does not include sequences of LT protein other than the above-defined truncated LT proteins. Therefore, the fusion protein never contains the full-length sequence of the native form of the LT antigen.

Amino acid sequence identity is defined as the percentage of amino acid residues in the variant sequence that are identical with the amino acid residues in the reference sequence after aligning the sequences and if necessary introducing gaps to achieve the maximum percent sequence identity, and not considering any conservative substitution as part of the sequence identity. Sequence identity may be determined in a global manner over the full length of the longest of the sequences being compared which may be the full length of the variant sequence or query sequence, or the full length of the reference sequence.

In practice, global sequence alignment can be achieved and percent identity can be determined by pairwise sequence alignment using algorithms that create an end-to-end optimal alignment (including gaps) of the sequences to be aligned, such as the Needleman-Wunsch algorithm (Needleman et al, J. Mol. Biol. (1970) 48: 444). The EMBOSS Needle tool uses the Needleman-Wunsch algorithm to read two input sequences (either amino acid or nucleotide sequences) and write their optimal global sequence alignment. This tool and others are accessible on the European Bioinformatics Institute (EBI) web site at http://www.ebi.ac.uk/services and may be used using the default settings.

The IT truncated form useful in the context of the present invention is expressed as a fusion protein which comprises at least one secretion peptide signal able to direct said fusion protein to the type 3 secretion system of the T3SS bacterial vector of the present invention.

The fusion protein comprises an N-terminal moiety which is the secretion peptide signal.

By “secretion peptide signal able to direct the fusion protein to the type 3 secretion system”, it is meant a peptide that contains information enabling the fusion protein to be targeted or addressed to the type 3 secretion system and to be secreted from the bacterial vector by the type 3 secretion system of the T3SS bacterial vector. This secretion peptide signal allows the system to distinguish the T3SS-transferred proteins (i.e. the fusion protein) from any other proteins of the interior of the bacterial vector (such as the cytosol of the bacterial vector). The function of the secretion peptide signal may be assessed by a secretion test as described in the example.

In one embodiment, the secretion peptide signal is the N-terminal moiety of a T3SS exotoxin (herein after called N-ter Exo).

The T3SS exotoxin may be selected from the group consisting of the N-terminal moiety of a Pseudomonas exotoxin e.g., the Pseudomonas exoS or exoT gene product (ExoS or ExoT), the N-terminal moiety of a Salmonella exotoxin, the N-terminal moiety of a Shigella exotoxin, the N-terminal moiety of a Yersinia exotoxin.

One skilled in the art knows how to choose the secretion peptide signal according to the bacterial vector of the invention (Krall R et al, J Biol Chem. 2004 Jan. 23; 279(4):2747-53. Epub 2003 Nov. 3.)

In a particular embodiment, the fusion protein comprises an N-terminal moiety which is the N-terminal moiety of a Pseudomonas exotoxin e.g., the Pseudomonas exoS or exoT gene product (herein after called ExoS or ExoT). While such a fusion protein can be expressed in any T3SS bacterial vector, it is convenient to express it in a Pseudomonas bacterial vector.

The amino acid sequence of the N-terminal moiety of the Pseudomonas exoS gene product (N-ter ExoS) is shown in SEQ ID NO: 2. It starts with the amino acid in position 1 (Met) of SEQ ID NO: 2 and ends with the amino acids in any one of the positions 15 to 129 of SEQ ID NO: 2, advantageously ends with the amino acid in any one of the positions 15 to 70 of SEQ ID NO: 2, more advantageously ends with the amino acid in any one of the positions 30 to 60 of SEQ ID NO: 2. An example of a useful N-ter ExoS sequence used as secretion peptide signal starts with the amino acid in position 1 (Met) of the SEQ ID NO: 2 and ends with the amino acid in position 54 of the SEQ ID NO: 2. The secretion peptide signal N-ter ExoS (1-54) is herein after referred to as S54.

The amino acid sequence of the N-terminal moiety of the Pseudomonas exoT gene product (N-ter ExoT) is shown in SEQ ID NO: 3. It starts with the amino acid in position 1 (Met) of SEQ ID NO: 3 and ends with the amino acid in any one of the positions 15 to 129 of SEQ ID NO: 3, advantageously ends with the amino acid in any one of the positions 15 to 70 of SEQ ID NO: 3, more advantageously ends with the amino acids in any one of the positions 30 to 60 of SEQ ID NO: 3. An example of a useful N-ter ExoT sequence, as a secretion peptide signal, starts with the amino acid in position 1 (Met) of SEQ ID NO: 3 and ends with the amino acid in position 54 of SEQ ID NO: 3. The secretion peptide signal N-ter ExoT (1-54) is herein after referred to as T54.

In a particular embodiment, the fusion protein can also comprise a moiety able to promote antigen-specific, cell-mediated immunity upon direct antigen delivery to host cells so that the induction of LT-specific CD8 T-cells be triggered. The term “direct antigen delivery” excludes antigen neosynthesis subsequent to nucleotide transfer. A number of proteins or peptides are already known in the art to promote cell-mediated immunity upon direct antigen delivery to host cells. As a matter of example, one may cite the Pan-HLA-DR-binding epitopes (Alexander et al, Immunity (1994) 1 (9): 751) (hereinafter called PADRE epitope). PADRE epitopes are known to enhance CD8 effectors via induction of a CD4+ helper T-cell response which in turn helps inducing a strong CD8+ cytotoxic T-cell response. An example of a PADRE epitope is the peptide having the amino acid sequence as shown in SEQ ID NO: 4.

In a preferred embodiment, the PADRE epitope is fused in frame between the secretion peptide signal and any one of the truncated LT forms as defined in the invention.

In another embodiment, the PADRE epitope is fused in frame after any one of the truncated LT forms as defined in the invention. In this case, the PADRE epitope is the C-terminal end of the fusion protein.

In another embodiment, wherein the fusion protein comprising (i) the N-terminal moiety of a T3SS exotoxin (N-ter Exo) e.g., N-ter ExoS or N-ter ExoT, and (ii) any one of the LT truncated forms as defined in the invention, the N-terminal amino acid of the PADRE peptide can be conveniently fused to the C-terminal amino acid of the N-ter Exo (e.g., N-ter ExoS or N-ter ExoT), and the C-terminal amino acid of the PADRE peptide can be conveniently fused to the N-terminal amino acid of any one of the LT truncated forms as defined in the invention, thereby leading to the fusion protein called N-ter Exo-PADRE-truncated LT, in particular leading to the fusion proteins called N-ter ExoS-PADRE-truncated LT and N-ter ExoT-PADRE-truncated LT.

In another embodiment, wherein the fusion protein comprising (i) the N-terminal moiety of a T3SS exotoxin (N-ter Exo) e.g., N-ter ExoS or N-ter ExoT, and (ii) any one of the LT truncated forms as defined in the invention, the N-terminal amino acid of the PADRE peptide can be conveniently fused to the C-terminal amino acid of any one of said LT truncated forms as defined in the invention, thereby leading to the fusion protein called N-ter Exo-truncated LT-PADRE, in particular leading to the fusion proteins called N-ter ExoS-truncated LT-PADRE and N-ter ExoT-truncated LT-PADRE.

For clarity's sake, in the context of the bacterial vector technology, it is indicated that there is no need to fuse (to tag) the LT truncated form with a moiety, such as a caireticulin (CRT) tag, able to enhance peptide presentation to MHC class I molecules.

According to a preferred embodiment, the fusion protein comprises, in particular consists (essentially) of (i) a secretion peptide signal as defined in the invention, (ii) a PADRE epitope as defined in the invention, (iii) a LT truncated form as defined in the invention.

According to another preferred embodiment, the fusion protein consists exclusively of (i) a secretion peptide signal as defined in the invention, (ii) a PADRE epitope as defined in the invention, (iii) a LT truncated form as defined in the invention.

All the variants described for the LT truncated form, all variants described for the secretion peptide signal apply to above-preferred embodiments and can be combined together with or without PADRE epitope. Accordingly, useful fusion proteins used in the context of the invention include the N-ter ExoS-truncated IT, N-ter ExoS-PADRE-truncated LT, N-ter ExoT-truncated LT and N-ter-ExoT-PADRE-truncated LT fusion proteins (non-limiting citations).

Another object of the invention concerns an expression cassette, wherein a nucleotide sequence encoding any one of the fusion protein as described in the invention is placed under the control of a promoter.

When the bacterial vector is a bacterium, then it may conveniently comprise an expression cassette, wherein a nucleotide sequence encoding any one of the fusion proteins as described in the invention is placed under the control of a promoter. In a particular embodiment, the promoter can be a promoter inducible by ExsA, such as a promoter of a gene encoding a T3SS exotoxin or a T3SS protein (globally referred to as T3SS promoters). Indeed, in natural conditions, the activity of the genes encoding the T3SS exotoxins (e.g., exoS, exoT, exoU, exoY) and that of the genes encoding the proteins constitutive of T3SS are modulated in trans by the ExsA transcription activator protein. In a particular embodiment, the promoter inducible by ExsA can be the Pseudomonas exoS or exoT promoter, especially when the secretion peptide signal of the fusion proteins as defined in the invention is the fragment of the N-terminal moiety of the Pseudomonas exoS or exoT gene product (N-ter ExoS or N-ter ExoT) respectively. Accordingly, the nucleotide sequence encoding the fusion protein as defined in the invention, such as the N-ter ExoS-truncated IT or N-ter ExoS-PADRE-truncated LT fusion protein, may be placed under the control of the exoS promoter. In a similar manner, the nucleotide sequence encoding a fusion protein as defined in the invention such as the N-ter ExoT-truncated LT or N-ter ExoT-PADRE-truncated LT fusion protein is placed under the control of the exoT promoter.

In a particular embodiment, when the bacterial vector is a bacterium, then it is able to express both the T3SS complex and the fusion protein as defined in the invention upon a single induction event. To this end, this bacterial vector comprising a first expression cassette, wherein a nucleotide sequence encoding the fusion protein as defined in the invention is placed under the control of an ExsA-inducible promoter, may also comprise a second expression cassette wherein a nucleotide sequence encoding ExsA is placed under the control of an inducible promoter e.g. a strongly-inducible promoter. The inducible promoter for ExsA expression may be a reagent-inducible promoter such as a promoter inducible by isopropyl-beta-D-thiogalactopyranoside (IPTG) i.e., the promoter of the lactose operon; in the absence of IPTG, the promoter is repressed. Alternatively, the inducible promoter may be a promoter inducible by a change in culture conditions other than the addition of a reagent, such as a change in temperature (heat-inducible promoter).

When the bacterial vector is a bacterium, it may have been transformed with the expression vector containing the cassette as described hereinabove. It is understood that all transformation techniques known to those skilled in the art can be used such as electroporation, triparental conjugation, bacteriophage transfection, etc. Accordingly, the invention also relates to a process of producing a T3SS bacterial vector being of bacterium, according to the invention and comprising (i) a first expression cassette comprising a sequence encoding a fusion protein as defined in the invention placed under the control of a promoter inducible by ExsA and (ii) a second expression cassette comprising a sequence encoding ExsA placed under the control of an inducible promoter; said process comprising:
(a) Culturing the bacterial vector in a culture medium, under conditions that repress ExsA expression;
(b) Inducing ExsA expression by a change in culture conditions, advantageously when the culture of the bacterial vector is in exponential phase i.e., mid-exponential phase;
(c) Optionally adding, a Ca2+ chelator (if not already present in the culture); and
(d) Recovering the bacterial vector at the end of the culture.

Addition of a Ca2+ chelator such as ethylene glycol tetraacetic acid (EGTA) or Ethylene diamine tetraacetic acid (EDTA) is advantageous in that it activates the expressed T3SS.

In a particular embodiment of the process according to the invention, the second expression cassette comprises a sequence encoding ExsA placed under the control of an IPTG-inducible promoter; and the process comprises:
(a) Culturing the bacterial vector in the absence of IPTG;
(b) Inducing ExsA expression by addition of IPTG, advantageously when the culture of the bacterial vector is in exponential phase i.e., mid-exponential phase;
(c) Optionally adding, a Ca2+ chelator (if not already present in the culture); and
(d) Recovering the bacterial vector at the end of the culture.

Advantageously, when the bacterial vector according to the invention is a bacterium, the expression cassette encoding the fusion protein as defined in the invention and/or the expression cassette encoding ExsA can be inserted into the chromosome of said bacterial vector or inserted into a self-replicative vector e.g., a plasmid which may advantageously be a multicopy plasmid.

Advantageously, when the bacterial vector is a bacterium, the maintenance in the bacterial vector of the self-replicative vector may be achieved by inserting into the self-replicative vector an expression cassette for the constitutive expression of an essential protein for the bacterial vector, such as DapD (2,3,4,5-tetrahydropyridine-2,6-dicarboxylate N-succinyltransferase), while the gene encoding this essential protein and naturally present in the bacterial genome has been inactivated e.g., by deletion from the bacterial genome. In this way, only the maintenance of the self-replicative vector in the bacteria guarantees the bacterial survival.

In an advantageous embodiment, when the bacterial vector according to the invention is a bacterium, the bacterial vector according to the invention is unable to substantially replicate in a host organism e.g., a mammal. To this end, the bacterial vector can conveniently be (i) live attenuated or (ii) sensitive to psoralen-induced cross-link upon exposure to long wavelength UVA light, thereby able to give a killed but metabolically active (KBMA) culture of the bacterial vector (upon appropriate photochemical treatment).

Live attenuation can conveniently be achieved by inactivating genes involved in particular metabolic pathways or critical virulence factors, in particular by non-reverting deletions. Aromatic amino acids such as tryptophan (Trp) do not exist in a free form in upper organisms. Accordingly, a bacterial vector auxotroph for such an amino acid cannot efficiently replicate in these organisms. A convenient bacterial vector auxotroph for aromatic amino acids such as Trp, may be a bacterial vector bearing a mutation inactivating the aroA gene which encodes the 3-phosphoshikimate 1-carboxyvinyltransferase which is a key enzyme in aromatic synthesis; and thereby lowering the bacterial vector ability for in vivo replication and pathogenicity. Such a mutation may be a ΔaroA deletion. As a matter of example, it is referred to ΔaroA Pseudomonas bacteria described in Epaulard et al, Clin. Vacc. Immunol. (2008) 15 (2): 308, such as CHA-OA (ΔexoS ΔexoT ΔaroA Δorf1) and CHA-OAL (ΔexoS ΔexoT ΔaroA Δorf1 Δlasl), and CLIN-1 (ΔexoS ΔexoT ΔaroA Δorf1 Δlasl, adapted for growth in a chemically defined medium as described in WHO 2013/087667). Such auxotroph strain may be available at the Collection Nationale de Culture de Microrganismes (CNCM, Institut Pasteur) under the name CLIN-01 and referenced I-4564 (deposited on Dec. 1, 2011 under the Budapest treaty by the University Joseph Fourier (Grenoble, FR) in relationship with WO 2013/087667. Alternatively, live attenuation can conveniently be achieved by UVA-light induced cross-link in the bacterial chromosome following psoralen treatment of a bacterial vector bearing inactivated uvr genes (A and B), which respectively encode the exonucleotidase A and B subunits which are required for nucleotide excision repair. Such a bacterial vector is therefore unable to substantially replicate after a photochemical treatment comprising adding psoralen to a culture of a bacterial vector, culturing the bacterial vector for an additional hour and then submitting the psoralen-treated culture to UVA irradiation. Addition of appropriate doses of psoralen and UVA, to be easily determined by a skilled man, results in a killed but metabolically active (KBMA) culture. By extension of language, the bacterial vector is itself labeled KBMA. As a matter of example, it is referred to the KBMA Pseudomonas bacterial vector described in Le Gouëllec et al, Mol. Therapy (2013) 21 (5): 1076 or WO 2013/087667 (KBMA CHA-OST ΔexoS ΔexoT ΔuvrAB), which also provides for technical details relating to the photochemical treatment.

In yet another particular embodiment, the bacterial vector according to the invention retains sensitivity to antibiotics.

In one embodiment, the T3SS bacterial vector is a bacterium belonging to Pseudomonas genus selected from the group consisting in CHA-003 as defined above, CHA-OA as defined above, CHA-OAL as defined above, CHA-OST as defined above and CLIN-01 as defined above. More particularly, the T3SS bacterial vector is the CHA-OST Pseudomonas bacterium.

Another object of the present invention concerns a fusion protein which comprises from its N-terminal end to its C-terminal end and fused in frame:
at least one secretion peptide signal able to direct said fusion protein to the type 3 secretion system of a bacterial vector, when said fusion protein is in a bacterial vector owning a type 3 secretion system, and
one truncated form of the Large T (LT) antigen of the Merkel Cell Polyomavirus (MCPyV) which has an amino acid sequence having at least 80% identity with one of the amino acid sequences shown in SEQ ID NO: 1 which start with an amino acid in any one of positions 1 to 5 and end with the amino acid in any one of the positions 210 to 469.

In a preferred embodiment, said fusion protein does not bind to the human retinoblastoma protein.

All features of the fusion protein able to be expressed, secreted and transferred by the T3SS bacterial vector as defined in the invention described above in connection with the T3SS bacterial vector, apply also for the invention related to the fusion protein.

By “fusion protein is in a bacterial vector”, it is meant that the fusion protein is inside the bacterial vector. The fusion protein may be expressed by the bacterial vector.

In a particular embodiment, the truncated form of the LT antigen of the MCPyV of the fusion protein has:
i. an amino acid sequence having at least 80% identity with the amino acid sequence shown on SEQ ID NO: 1 which start with the amino acid in position 2 and end with the amino acid in position 215, and, preferably, provided that said truncated form of the LT antigen of the MCPyV does not bind to the human retinoblastoma protein, or
ii. an amino acid sequence having at least 80% identity with the amino acid sequence shown on SEQ ID NO: 1 which start with the amino acid in position 2 and end with the amino acid in position 270, and, preferably, provided that said truncated form of the LT antigen of the MCPyV does not bind to the human retinoblastoma protein, or
iii. one of the amino acid sequences shown in SEQ ID NO: 1 which starts with the amino acid in any one of positions 1 to 5, advantageously in position 2, and ends with the amino acid in any one of positions 210 to 215, so that said truncated form of the LT antigen of the MCPyV does not bind to the retinoblastoma protein; or
iv. one of the amino acid sequences shown in SEQ ID NO: 1 which starts with the amino acid in any one of positions 1 to 5, advantageously in position 2, and ends with the amino acid in any one of positions 216 to 270, which is further mutated in the human retinoblastoma protein binding site located from positions 212 to 216 in SEQ ID NO: 1, so that said truncated form of the LT antigen of the MCPyV does not bind to the human retinoblastoma protein.

In a particular embodiment, the amino acid sequence of the LT truncated form of the fusion protein according to the invention has an amino acid sequence shown in SEQ ID NO: 1 which starts with the amino acid in any one of positions 1 to 5, advantageously in position 2, and ends with the amino acid in position 215.

In a particular embodiment, the amino acid sequence of the LT truncated form of the fusion protein according to the invention has:
one of the amino acid sequences shown in SEQ ID NO: 1 which starts with the amino acid in any one of positions 1 to 5, advantageously in position 2, and ends with the amino acid in any one of positions 250 to 260, advantageously in position 259, which is further mutated in the human retinoblastoma protein binding site located from positions 212 to 216 in SEQ ID NO: 1, so that said LT truncated form does not bind to the human retinoblastoma protein; or
an amino acid sequence having at least 85% identity with the amino acid sequence shown in SEQ ID NO: 1 which starts with the amino acid in position 2 and ends with the amino acid in position 259 and, preferably, provided that said LT truncated form does not bind to the human retinoblastoma protein.

In a particular embodiment, the amino acid sequence of the LT truncated form of the fusion protein according to the invention has at least 80%, 85%, 90%, 95%, or advantageously 100% identity, with the amino acid sequence shown in SEQ ID NO: 1 which starts with the amino acid in position 2, and ends with the amino acid in position 469 and, preferably, provided that said LT truncated form does not bind to the human retinoblastoma protein is not able to bind the pRB.

In another particular embodiment, the amino acid sequence of the LT truncated form of the fusion protein according to the invention has at least 80%, 85%, 90%, 95%, or advantageously 100% identity, with the amino acid sequence shown in SEQ ID NO: 1 which starts with the amino acid in position 2, and ends with the amino acid in position 215 and, preferably, provided that said LT truncated form does not bind to the human retinoblastoma protein. A typical example of this is LT(2-215) by reference to SEQ ID NO: 1.

In another particular embodiment, the LT truncated form of the fusion protein according to the invention is defined by an amino acid sequence consisting of the amino acid sequence shown in SEQ ID NO: 1 which starts with the amino acid in any one of positions 1 to 5, advantageously in position 2, and ends with the amino acid in any one of positions 216, 220, 230, 240 or 250 to 270, advantageously in any one of positions 250 to 260, preferably in position 259; which is further mutated in the human retinoblastoma protein binding site (RBS) located from positions 212 to 216 in SEQ ID NO: 1, so that said LT truncated form is unable to bind the human retinoblastoma protein (pRb) and in particular so that said LT truncated form has a non-functional RBS site.

Alternatively, the amino acid sequence of the LT truncated form of the fusion protein according to the invention has:
at least 80%, 85%, 90% or 95% identity, with the amino acid sequence shown in SEQ ID NO: 1 which starts with the amino acid in position 2 and ends with the amino acid in position 270; or
at least 85%, 90%, 95%, 97% or 99% identity with the amino acid sequence shown in SEQ ID NO: 1 which starts with the amino acid in position 2 and ends with the amino acid in position 259;
   and, preferably, providing that said LT truncated form does not bind to the human retinoblastoma protein is unable to bind the human retinoblastoma protein (pRb), in particular that said LT truncated form has a non-functional RBS site.

In one embodiment, a non-functional RBS may be obtained by introducing mutation in RBS of the truncated LT antigen when said truncated LT antigen contains at least the first 216 amino acids of the native form of the LT antigen. The RBS located from positions 212 to 216 in SEQ ID NO: 1 is mutated so that it is rendered non-functional, leading to an LT truncated form and a fusion protein unable to bind pRb. While the RBS may be mutated in any of positions 212 to 216, it is preferred to mutate in position 212, 214 or 216 of SEQ ID NO: 1. As a matter of example, a useful mutation is a mutation substituting the glutamic acid in position 216 by another amino acid which is not glutamine, acid aspartic or asparagine. Advantageously, the substitution amino acid is a neutral or positively-charged amino acid. A useful neutral amino acid may be alanine, valine, leucine, isoleucine, methionine, phenylalanine, threonine or tryptophan. A useful positively-charged amino acid may be arginine, histidine or lysine, this latter being preferred. As a matter of example, a useful mutation is a mutation substituting the glutamic acid residue in position 216 by lysine residue. An example of truncated form of LT having a non-functional RBS is a truncated form of LT having an amino acid sequence shown in SEQ ID NO: 1 which starts with an amino acid in any one of the positions 1 to 5, advantageously in position 2, and ends with the amino acid in any one of the positions 210 to 469 and is further mutated in the RBS located from position 212 to 216 in SEQ ID NO: 1. Accordingly, a typical example of an LT truncated form having a non-function RBS site (i.e a LT truncated and mutated form) is LT(2-259, E216→K) by reference to SEQ ID NO: 1. In another embodiment, an LT truncated form having a non-functional RBS may be obtained by substituting any one of the amino acids located from position 212 to the 216 in SEQ ID NO: 1 by a codon stop. Accordingly, a typical example of an LT truncated form having a non-functional RBS is LT(2-215).

For the sake of brevity, the term “LT truncated forms” will herein after include all the LT truncated forms within the scope of the invention, including the LT truncated and mutated forms and the LT truncated form having no RBS site such as LT truncated forms having an amino acid sequence consisting of the amino acid sequence shown in SEQ ID NO: 1 which starts with the amino acid in any one of positions 1 to 5, advantageously in position 2, and ends with the amino acid in any one of positions 210 or 211.

In a preferred embodiment, the fusion protein comprises the LT truncated form, as the sole antigen comprising MCPyV epitopes able to be expressed, secreted and transferred, preferably translocated by the bacterial vector according to the invention. That means that the fusion protein of the invention does not include sequences of LT protein other than the above-defined truncated LT proteins. Therefore, the fusion protein of the invention never contains the full-length sequence of the native form of the LT antigen.

The fusion protein of the present invention comprises at least one secretion peptide signal able to direct said fusion protein to the type 3 secretion system of a T3SS bacterial vector, as defined in the invention. All features related to the secretion peptide signal that have been described in relation with the bacterial vector apply here.

In a particular embodiment, the fusion protein can also comprise a moiety able to promote antigen-specific, cell-mediated immunity upon direct antigen delivery to host cells so that the induction of LT-specific CD8 T-cells may be triggered. All features related to said moiety that have been described in relation with the bacterial vector apply here.

For clarity's sake, it is indicated that there is no need to fuse (to tag) the LT truncated form with a moiety, such as a calreticulin (CRT) tag, able to enhance peptide presentation to MHC class I molecules.

According to a preferred embodiment, the fusion protein comprises, in particular consists (essentially) of (i) a secretion peptide signal as defined in the invention, (ii) a PADRE epitope as defined in the invention, (iii) a LT truncated form as defined in the invention. According to another preferred embodiment, the fusion protein consists exclusively of (i) a secretion peptide signal as defined in the invention, (ii) a PADRE epitope as defined in the invention, (iii) a LT truncated form as defined in the invention.

All the variants described for the LT truncated form, and all the variants described for the secretion peptide signal apply to above-preferred embodiments and can be combined together with or without PADRE epitope.

In a preferred embodiment, the fusion protein is the S54-PADRE-LT(2-259, E216→K) protein or S54-PADRE-LT(2-215) protein, wherein the S54 moiety, PADRE epitope, LT(2-259,E216→K) and LT(2-215) are as described in the invention.

Yet another aspect of the invention relates to an isolated polynucleotide encoding the fusion protein of the invention. The polynucleotide is a synthetic or recombinant DNA either single- or double-stranded.

Preferably, the isolated polynucleotide comprises a coding sequence which is optimized for the T3SS bacterial vector, selected for the secretion, transfer, preferably translocation, of the fusion protein, owning a type 3 secretion system in which the fusion protein of the invention is expressed. All features related to the T3SS bacterial vector as defined in the invention, apply here. In a preferred embodiment, the T3SS vector is a bacterium belonging to the Pseudomonas strain. As an example, a nucleic acid codon-optimized if at least one codon in the isolated polynucleotide is replaced with codon that is more frequently used by Pseudomonas for that amino acid than the codon in the original sequence. Said isolated polynucleotide may be introduced in an expression cassette in order to be expressed in a T3SS bacterial vector as defined in the invention.

The T3SS bacterial vector as defined in the invention is genetically modified for inducing an expression cassette for the expression of the fusion protein.

In a preferred embodiment of the bacterial vector as defined in the invention, said polynucleotide is inserted under the control of promoter into an expression cassette such as a plasmid. The plasmid is capable of expressing said polynucleotide when transfected or transformed into a bacterial vector owning a type 3 secretion system. Plasmids are known in the art, and a number of them are commercially available, but it is also possible to construct or modify them using genetic manipulation techniques.

In a particular embodiment, the promoter can be a promoter inducible by ExsA, such as a promoter of a gene encoding a T3SS exotoxin or a T3SS protein (globally referred to as T3SS promoters). In a particular embodiment, the promoter inducible by ExsA can be the Pseudomonas exoS or exoT promoter, especially when the fusion protein of the invention comprises an N-terminal moiety which is the fragment of the N-terminal moiety of the Pseudomonas exoS or exoT gene product (N-ter ExoS or N-ter ExoT) respectively. Accordingly, the nucleotide sequence encoding a fusion protein such as the N-ter ExoS-truncated LT or N-ter ExoS-PADRE-truncated LT fusion product may be placed under the control of the exoS promoter. In a similar manner, the nucleotide sequence encoding a fusion protein such as the N-ter ExoT-truncated LT or N-ter ExoT-PADRE-truncated LT fusion product, is placed under the control of the exoT promoter.

The bacterial vector as described in the invention may be transformed with the plasmid described hereinabove.

It is understood that all transformation techniques known to those skilled in the art can be used such as electroporation, triparental conjugation, bacteriophage transfection, etc.

Under another aspect, the invention relates to:
(I) A pharmaceutical composition comprising a bacterial vector according to the invention;
(II) A bacterial vector according to the invention for use as a medicament;
(III) A bacterial vector according to the invention for use as a medicament in the treatment of cancer or for the manufacture of a medicament for the treatment of cancer;
(IV) A bacterial vector according to the invention for use in a method of combating or preventing MCPyV infection, and, in particular, as a medicament;
(V) The use of a bacterial vector according to the invention, for the manufacture of a medicament for combating or preventing MCPyV infection; and
(VI) A method of combating or preventing MCPyV infection which comprises administering to a patient in need, a bacterial vector according to the invention or a pharmaceutical composition comprising a bacterial vector according to the invention.

MCPyV infection can be combated or prevented by promoting in a patient a CD8+ immune response against cells infected with MCPyV, including (i) cells expressing whole or part of the MCPyV LT antigen, and (ii) cells (a) expressing whole or part of the MCPyV LT antigen and (b) in the genome of which is integrated an MCPyV nucleotide sequence encoding said whole or part of the LT antigen. The immune response against cells infected with MCPyV is advantageously LT-specific.

By “CD8+ immune response” is meant an immune response characterized by the induction of CD8 T-cells, in particular CD8 cytotoxic T-cells. CD8 T-cells express the CD8 glycoprotein at their membrane surface. The induction of CD8 cytotoxic T-cells i.e., LT-specific CD8 cytotoxic T-cells, is effective in significantly reducing the amount of cells expressing whole or part of the LT antigen.

MCPyV infection may be asymptomatic or not. Accordingly, cells infected with MCPyV may be in a tumoral state or not. In asymptomatic patients, non-tumoral cells infected with MCPyV, in particular those expressing whole or part of the LT antigen, are nevertheless indicative of a pre-tumoral status and it is therefore desirable to eliminate them because the patients are indeed at risk of developing a tumor. In addition to this, cells (a) expressing whole or part of the MCPyV LT antigen and (b) in the genome of which is integrated an MCPyV nucleotide sequence encoding said whole or part of the LT antigen are indicative of a higher risk of developing a tumor.

Accordingly, the bacterial vector according to the invention or the pharmaceutical composition comprising a bacterial vector according to the invention, can also be for use in:
(i) A method of preventing tumor appearance in asymptomatic patients infected with MCPyV;
(ii) A method of reducing the growth or propagation of a tumor in a patient suffering from a tumoral disorder characterized by the presence of tumoral cells expressing the LT antigen of MCPyV;
(iii) A method of treating a tumoral disorder characterized by the presence of tumoral cells expressing the LT antigen of MCPyV.

Accordingly, the invention also relates to any one of methods (i) to (iii) specified above, wherein the bacterial vector according to the invention is administered to a patient in need.

Accordingly, the invention also relates to any one of methods (i) to (iii) specified above, wherein the pharmaceutical composition comprising the bacterial vector according to the invention is administered to a patient in need.

MCPyV infection, asymptomatic or not, may be revealed by PCR amplification of viral DNA from a blood sampling. PCR amplification may be conventionally achieved by a man skilled in the art. Amplification of MCPyV DNA may be achieved using PCR sense and antisense primers respectively shown in SEQ ID NO: 11 and 12.

Alternatively, when possible, cell sampling may be achieved in asymptomatic patients associated with a particular risk factor e.g., a broncho-alveolar lavage in asymptomatic smokers or biopsy in asymptomatic patients with family antecedents for a particular type of cancer. This cell sampling (e.g., biopsy) followed by the search for LT expression and viral DNA integration into the cell genome, will indicate the risk level of developing a tumor.

LT expression may be revealed i.e., by (i) detecting the presence of the LT antigen in the cell extract by Western blot or (ii) by immunohistochemistry. Those techniques are known in the art and may conventionally be achieved by a man skilled in the art. Those techniques require the use of a monoclonal antibody generated against MCPyV LT antigen residues 1-260 such as monoclonal antibody CM2B4 supplied by Santa Cruz Biotech Inc. under reference sc-136172 or monoclonal antibody Ab3 described in Rodig et al, J. Clin. Invest. (2012) 122: 4645.

Viral DNA integration may be revealed by isolating the cell DNA and detecting the viral DNA by PCR amplification using PCR sense and antisense primers respectively shown in SEQ ID NO: 11 and 12.

In cancer patients, the MCPyV infection may also be detected upon cell sampling e.g., biopsy or blood sampling followed by the showing of LT expression and viral DNA integration into the cell genome, using the methods reported herein above.

Tumoral disorders intended for prevention or treatment, may be i.e., any one of Merkel cell carcinoma, neuro-endocrine cutaneous carcinoma, squamous cell carcinoma, basal cell carcinoma, Bowen's disease, cutaneous B-cell lymphoma, cutaneous T-cell lymphoma, chronic lymphocytic leukemia, small lymphocytic lymphoma, human central nervous system tumors, chronic lymphotic leukemia, glioblastoma, cervical carcinoma, large cell lung carcinoma, lung adenocarcinoma or small cell lung cancer.

A pharmaceutical composition according to the invention i.e., be a vaccinal composition, comprises a bacterial vector according to the invention together with a pharmaceutically acceptable diluent or carrier. Accordingly, the bacterial vector can be formulated for parenteral administration e.g., in a pharmaceutically acceptable diluent. Alternatively, the bacterial vector can be lyophilized for storage and extemporaneously diluted in a pharmaceutically acceptable diluent, prior use.

The method of combating or preventing MCPyV infection or any one of the methods (i) to (iii) according to the invention can comprise parenterally, i.e., subcutaneously, intradermally or intravenously, administering the bacterial vector of the invention or a pharmaceutical composition of the invention to a patient in need. Conveniently, a dose of a bacterial vector of the invention may be administered one or several times a day, week or month interval. As illustrative example only, it is indicated that a dose may comprise from 1×10⁶ to 1×10¹⁰ colony-forming units (cfu) when a bacterial vector is administrated.

As a matter of non-limiting example, a bacterial vector according to the invention may be a Pseudomonas bacterial vector unable to express ExoS, ExoT and ExoU and able to express, secrete and transfer, preferably translocate via the T3SS complex, the fusion protein S54-PADRE-LT(2-259, E216→K) or S54-PADRE-LT(2-215), wherein the S54 moiety, PADRE epitope, LT(2-259,E216→K) and LT(2-215) are as described herein above. This is further illustrated in the Experimental Section below.

### FIGURES

FIG. 1 is a schematic representation of plasmid pEAI-S54 described in Wang et al, 2012.

FIG. 2 shows a Western blot analysis of the supernatant of an LT^{VAX} culture, using an LT-specific monoclonal antibody.

FIG. 3 shows the IFNg release (pg/ml) by NP68-specific CD8 T-cells co-cultivated with DCs previously incubated with LT^{VAX}, as measured by an ELISA assay.

FIG. 4 shows the IFNg release (pg/ml) by SIINFEKL-specific CD8 T-cells co-cultivated with B16 LT tumor cells, as measured by an ELISA assay.

FIG. 5 shows the tumor growth in B16 LT tumor cell-bearing mice treated with LT^{VAX}.

### EXPERIMENTALS

### Materials and Methods

### DNA Cloning

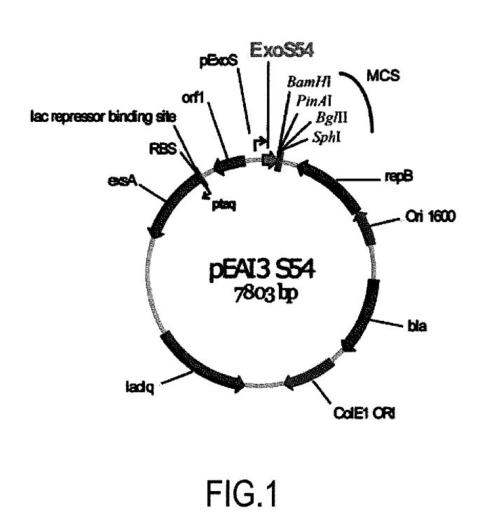

DNA sequence that encodes the LT protein of Merkel Cell Polyomavirus (MCPyV) as shown in SEQ ID NO: 1 was optimized in silico for expression in Pseudomonas aeruginosa. The DNA sequence that encodes for the LT truncated form LT(2-215) is shown in SEQ ID NO: 22. The DNA sequence that encodes for the LT truncated and mutated form LT(2-259,E216->K) is shown in SEQ ID NO: 7. These sequences were cloned in the monocistronic pEAI-S54-PADRE plasmid (Wang et al, 2012; FIG. 1) downstream the nucleotide PADRE sequence (SEQ ID NO: 9). This generated the plasmids pEAI-S54-PADRE-LT(2-215) and pEAI-S54-PADRE-LT(2-259, E216->K) able to express the fusion protein S54-PADRE-LT(2-215) and S54-PADRE-LT(2-259, E216->K), respectively.

DNA sequence (SEQ ID NO: 7) that encodes the amino acid sequence (2-259, E216->K) was also cloned in the same monocistronic pEAI-S54-PADRE plasmid downstream the nucleotide PADRE sequence (SEQ ID NO: 9) together with the sequence encoding the NP68 peptide (as shown in SEQ ID NO: 10). This generated the plasmid pEAI-S54-PADRE-LT(2-259, E216->K)-NP68 able to express the fusion protein S54-PADRE-LT(2-259,E216->K)-NP68. The NP68 peptide (SEQ ID NO 5) which corresponds to amino acids 362-378 of the nucleoprotein (NP) protein of the Influenza virus strain A/Nt/60/68, was used as a tag for monitoring the immune response against the expression product.

Cloned sequence was verified by DNA sequencing.

Plasmids pEAI-S54-PADRE-LT(2-215) and pEAI-S54-PADRE-LT(2-259, E216-K) have been used for the secretion assay. These plasmids encode for the fusion protein S54-PADRE-LT(2-215) and S54-PADRE-LT(2-259, E216->K) respectively.

The plasmid pEAI-S54-PADRE-LT(2-259, E216->K)-NP68 has been used for the generation of LT^{VAX} as this plasmid permitted the in vitro validation of antigen delivery thanks to the presence of NP68. This plasmid encodes for the fusion protein S54-PADRE-LT(2-259,E216->K)-NP68.

For the generation of the B16 LT tumor cells, the LT-IRES-GFP plasmid was constructed to express under the control of the EF1a promoter, (i) the LT(1-259, E216->K) tagged in C-ter by the ovalbumine peptide (257-264) SIINFEKL (SEQ ID NO: 6) and (ii) the Green Fluorescent Protein (GFP). In the pCDNA3.1 plasmid (Life Technologies) devoid of the CMV promoter, the following elements were inserted from 5′ to 3′: the EF1a promoter (SEQ ID NO: 25), the DNA sequence encoding LT(1-259, E216->K) (SEQ ID NO: 23), the DNA sequence encoding the SIINFEKL peptide, an Internal Ribosomal Entry Site (IRES; SEQ ID NO: 24) and the DNA sequence encoding GFP (SEQ ID NO: 21) in order to obtain the LT-IRES-GFP plasmid. The SIINFEKL peptide was used as a tag for monitoring the immune response against the B16 LT cell line.

### Generation of LT^{VAX}

The LT^{VAX} bacterial vector was generated by transforming the P. aeruginosa, attenuated strain CHA-OST (Epaulard et al, 2006) with the plasmids pEAI-S54-PADRE-LT(2-215), pEAI-S54-PADRE-LT(2-259, E-216->K) or the plasmid pEAI-S54-PADRE-LT(2-259, E216->K)-NP68. Briefly, the CHA-OST strain was incubated in Luria Bertani broth (Miller) at 37° C., 225 rpm agitation for 16 hours. 2 ml of bacterial culture were resuspended in 300 mM sucrose solution (Euromedex). After 2 washes, 1/10 of the bacterial population was incubated for 20 minutes at 4° C. with 100 ng of plasmid DNA. Electroporation was performed at 1.8 kV for 5 milli seconds. After a 1 hour incubation, at 37° C., 225 rpm in SOC (Super Optimal Catabolite) medium (Life Technologies), bacteria were plated on Pseudomonas isolation agar (PiA) medium (Gibco) supplemented with 300 μg/ml carbenicillin (Sigma).

### Culture of LT^{VAX}

LT^{VAX} was incubated at 37° C. with 300 rpm agitation, in a chemically defined medium based on the glucose minimal medium (M9) supplemented with magnesium and calcium, named MM9 medium (Le Gouëllec et al, 2013). Precisely, MM9 contains: extract of synthetic yeast without tryptophan 4 g/L (Sigma); FeSO₄ 0.4 g/L (Sigma); glucose 2.5 g/L (Euromedex); glycerol 1% (Euromedex); Citric acid 0.36 g/L (Euromedex); M9 Salts Medium 5× (Sigma).

The expression of the fusion protein S54-PADRE-LT(2-215), S54-PADRE-LT(2-259, E216-K) S54-PADRE-LT(2-259, E216->K)-NP68 and T3SS was induced by addition of isopropyl-beta-D-thiogalactopyranoside (IPTG) and the T3SS function was activated by Ca2+ chelation with ethylene glycol tetraacetic acid (EGTA). Briefly, LT^{VAX} was then diluted at Optical Density (OD₆₀₀) of 0.2 in MM9 medium supplemented with 300 μg/mL carbenicillin, 1.6 mM IPTG, 5 mM EGTA and 20 mM MgCl₂ (all from Euromedex), and incubated at 37° C. with 300 rpm agitation until OD₆₀₀ 1.6 was reached. LT^{VAX} was then resuspended in MM9 medium, ready for in vivo or in vitro use.

### Protein Secretion Assay

The LT^{VAX} culture was centrifuged at 13 000 g for 10 minutes. In order to precipitate proteins, the supernatant was incubated with 20% (v/v) trichloroacetic acid at 4° C. for 10 minutes, then centrifuged at 15000 g, 4° C. for 5 minutes and washed twice with acetone (Sigma), centrifuging at 15000 g for 5 minutes after each wash. Proteins were resuspended in 80 μl of denaturation buffer (0.5 M Tris-HCL, 0.6 M DTT, 10% SDS, 0.012% bromophenol blue, 15% glycerol) and migrated in SDS-PAGE in a 10% polyacrylamide gel (Ready Gels Precast Gel, Biorad) following manufacturer's instructions.

### Western Blot Analysis

Proteins were loaded in 12% acrylamide gel (Promega), and run in running buffer (Promega). Protein transfer was performed at 120 V for 60 minutes with the precast system (Promega). Anti-LT antibodies (2000-fold dilution, purchased from Santa Cruz Biotech; ref: sc-136172) and anti-mouse horse radish peroxidase secondary antibody (10⁴ dilution, from Dutscher) were used.

### Cell Lines and Primary Cells

B16F0 cells (ATCC-CRL-6322) and B16LT tumor cells were cultured in DMEM medium, supplemented with 10% FBS, penicillin (100 U/ml), streptomycin (100 U/ml), 1 mM glutamine, and 1 mM sodium pyruvate (all items from Life Technologies).

Primary dendritic cells (DCs) were obtained as follows: bone marrow from tibia and femurs of C57BL/6 mice was flushed and cultured in RPMI 1640 medium supplemented with 10% heat-inactivated foetal bovine serum (FBS), 2 mM L-glutamine, 10 mM HEPES buffer, 50 μg/ml gentamicin (all from Life Technologies) and 50 μM 2-Mercaptoethanol (Sigma-Aldrich). Red blood cells were lysed by resuspending pelleted cells in Tris-ammonium chloride (Life Technologies) for 2 minutes. Cells were then resuspended in culture medium and cultured for 9 days at 10⁶ cells/ml in six-well plate, at 37° C. and 7% CO₂, in culture medium supplemented with 200 ng/ml recombinant human Flt3 ligand (Amgen) for monocyte differentiation into DCs.

Activated, anti-NP68 T-cells were obtained from splenocytes of F5 transgenic mice (Mamalaki et al, 1993). Spleen was collected aseptically and splenocytes were cultured in DMEM supplemented with 6% FBS, 2 mM L-glutamine, 10 mM HEPES buffer, 50 μM 2-Mercaptoethanol, 10% recombinant IL-2 and 10 nM NP68 peptide (ProteoGenix). After 5 days of culture, cells were harvested and cultured for two additional days in culture medium, in the absence of peptide NP68.

Activated, anti-ovalbumine peptide SIINFEKL T-cells were obtained from splenocytes of OT1 transgenic mice (Hogquist et al, 1994; Clarke et al, 2000). Spleen was collected aseptically and splenocytes were cultured in DMEM supplemented with 6% FBS, 2 mM L-glutamine, 10 mM HEPES buffer, 50 μM 2-Mercaptoethanol and 10 nM SIINFEKL peptide (ProteoGenix). After 5-day culture, cells were harvested and cultured for two additional days in culture medium, in the absence of peptide SIINFEKL.

### In Vitro Validation of LT^{VAX} Antigen Delivery

Primary dendritic cells (DCs) were seeded in 96-well plate at 10⁵ cells/well in 100 μl of medium. DCs were co-incubated with LT^{VAX} or BacVac™ vector (Pseudomonas strain CHA-OST transformed with the pEAI-S54-PADRE plasmid) at multiplicity of infection of 0.1. After 1 hour, bacteria were removed by performing two washes with DC culture medium, and incubating DCs twice (30 minutes at each time) in medium enriched with gentamycin (Life Technologies). Activated, anti-NP68 T-cells were co-incubated with DCs at ratio 1:1 and incubated at 37° C., 7% CO₂. Co-culture supernatants were collected 16 hours later. IFNg levels measured in the co-culture supernatants were quantified by ELISA (ReD systems) following manufacturer's instructions.

### Generation of the B16 LT Tumor Cell Line

B16F0 tumor cells were transfected with the LT-IRES-GFP plasmid, using the jetPEI™ transfection reagent (PolyPlus) following manufacturer's instructions to give B16F0 tumor cells expressing LT(1-259, E216->K) (now called B16 LT tumor cells). Stable plasmid integration in the eukaryotic cell genome was obtained by incubating B16 LT tumor cells in medium supplemented with 10% FBS and 0.1 g/L G418 (Sigma). Surviving, GFP-positive tumor cell clones were isolated.

### Validation of the B16 LT GFP-Positive Cells for Use in the Tumor Model

B16 LT, GFP-positive tumor cells (and B16F0 tumor cells) were respectively co-seeded with activated, SIINFEKL-specific T-cells, at ratio 1:1 and co-incubated at 37° C., 7% CO₂. Co-culture supernatants were collected 16 hours later. IFNg levels were quantified by ELISA (ReD systems) following manufacturer's instructions.

### Tumor Challenge Experiment

Female C57BL/6j mice were purchased from Janvier S A (Le Genest-Saint-Isle, France) and kept under pathogen-free conditions in the animal facility of the University Joseph Fourier (Grenoble, France). Experiments were approved by the Animal Experiment Committee of the Region and were performed in accordance with institutional and national guidelines. 2×10⁵ B16 LT tumor cells were resuspended in PBS and administered subcutaneously into the flank of 6-8 week old mice. Mice were monitored for tumor appearance every 24 hours. LT^{VAX} injection was performed subcutaneously (5*10⁵ bacteria for the first two injections, then 10⁶ for the following injections) when tumor became palpable (5-7 days post inoculation) and then every three-four days throughout the experiment. Groups of 6 mice per condition were used. Tumor size was measured by using a caliper. Two measures were performed.

### Results

### LT^{VAX} Efficiently Secretes Fusion Proteins Comprising the Truncated Form of LT (2-215) or the Truncated Form of LT(2-259, E216→K)

LT^{VAX} is an immunotherapy product based on the BacVac™ technology (Epaulard 2006). LT^{VAX} was obtained by transforming a BacVac™ vector (CHA-OST strain of P. aeruginosa) with a plasmid encoding a fusion product of (i) the P. aeruginosa ExoS peptide (1-54), also called the S54 peptide, and (ii) a mutated, truncated form of the LT antigen of MCPyV, hereinafter called LT (2-259, E216→K). The E216→K mutation was introduced to inactivate RBS so that the LT product (in particular the fusion protein) encoded by the plasmid is substantially non-oncogenic. The encoding sequence was optimized for expression in P. aeruginosa. Alternatively, LT^{VAX} was obtained by transforming the BacVac™ vector with a plasmid encoding a fusion product of (i) the S54 peptide, and (ii) a mutated, truncated form of the LT antigen of MCPyV, hereinafter called LT (2-215) This truncation generates an RBS so that the LT product (in particular a fusion protein) encoded by the plasmid is substantially non-oncogenic. The encoding sequence was optimized for expression in P. aeruginosa.

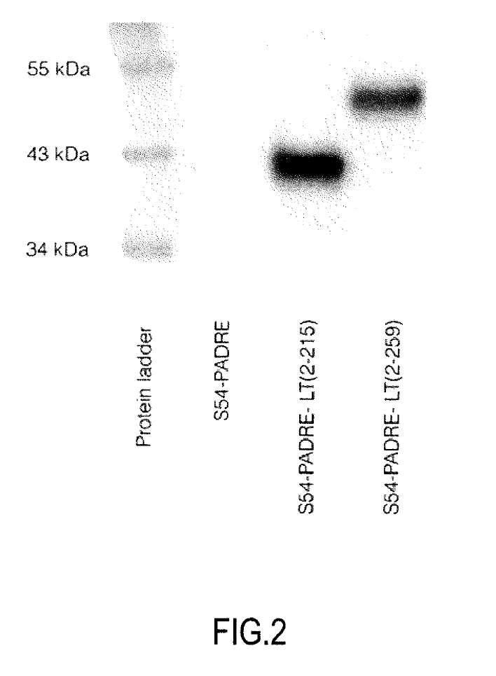

LT^{VAX} were cultured and incubated with (i) IPTG to express the exsA gene product which in turn activates the exoS promoter; and (ii) EGTA, to trigger delivery of the fusion protein S54-PADRE-LT(2-259, E216->K) or S54-PADRE-LT(2-215) via the T3SS. As shown in FIG. 2, Western blot analysis of the supernatant of an LT^{VAX} culture, using an LT-specific monoclonal antibody, demonstrates that the fusion protein comprising the LT (2-259, E216→K) and the LT(2-215) were efficiently expressed and secreted via the T3SS, thanks to the S54 peptide. Secretion of the fusion protein comprising the LT (2-259, E216→K) and the LT(2-215) were not observed in the control test (BacVac™ transformed with the pEAI-S54-PADRE plasmid).

Moreover, secretion tests as described above have also been performed with the following fusion proteins:
S54-LT(2-817) which corresponds to a fusion protein comprising the S54 peptide and the native form of the LT antigen of the MCPyV having the amino acid sequence shown in SEQ ID NO: 13 which starts to the amino acid in position 2 and ends with the amino acid in position 817. The nucleic sequence shown in SEQ ID NO: 14 which has been optimized for expression in P. aeruginosa has been used for the construction of said fusion protein. The nucleic sequence shown in SEQ ID NO: 8 which encodes for the S54 peptide has also been used for the construction of said fusion protein.
S54-PADRE-sT(2-186) which corresponds to a fusion protein comprising the S54 peptide, the PADRE epitope and the native form of the Small T (sT) antigen of the MCPyV having the amino acid sequence shown in SEQ ID NO: 15 which starts to the amino acid in position 2 and ends with the amino acid in position 186. The nucleic sequence shown in SEQ ID NO: 16 which has been optimized for expression in P. aeruginosa has been used for the construction of said fusion protein. The nucleic sequence shown in SEQ ID NO: 8 which encodes for the S54 peptide and the nucleic sequence SEQ ID NO: 9 which encodes for the PADRE epitope have also been used for the construction of said fusion protein.
S54-PADRE-sT(81-186) which corresponds to a fusion protein comprising the S54 peptide, the PADRE epitope and the sT truncated form having the amino acid sequence shown in SEQ ID NO: 15 which starts to the amino acid in position 81 and ends with the amino acid in position 186. The nucleic sequence shown in SEQ ID NO: 17 which has been optimized for expression in P. aeruginosa has been used for the construction of said fusion protein. The nucleic sequence shown in SEQ ID NO: 8 which encodes for the S54 peptide and the nucleic sequence SEQ ID NO: 9 which encodes for the PADRE epitope have also been used for the construction of said fusion protein.
S54-PADRE-sT(2-141) which corresponds to a fusion protein comprising the S54 peptide, the PADRE epitope and the sT truncated form having the amino acid sequence shown in SEQ ID NO: 15 which starts to the amino acid in position 2 and ends with the amino acid in position 141. The nucleic sequence shown in SEQ ID NO: 18 which has been optimized for expression in P. aeruginosa has been used for the construction of said fusion protein. The nucleic sequence shown in SEQ ID NO: 8 which encodes for the S54 peptide and the nucleic sequence SEQ ID NO: 9 which encodes for the PADRE epitope have also been used for the construction of said fusion protein.
S54-PADRE-sT(81-141) which corresponds to a fusion protein comprising the S54 peptide, the PADRE epitope and the sT truncated form having the amino acid sequence shown in SEQ ID NO: 15 which starts to the amino acid in position 81 and ends with the amino acid in position 141. The nucleic sequence shown in SEQ ID NO: 19 which has been optimized for expression in P. aeruginosa has been used for the construction of said fusion protein. The nucleic sequence shown in SEQ ID NO: 8 which encodes for the S54 peptide and the nucleic sequence SEQ ID NO: 9 which encodes for the PADRE epitope have also been used for the construction of said fusion protein.

The Small T (sT) antigen is an oncoprotein expressed by MCPyV. The first 79^{th} amino acid of sT antigen are identical to the first 79^{th} amino acid of LT antigen.

Said fusion proteins (S54-LT(2-817)), (S54-PADRE-sT(2-186)), (S54-PADRE-sT(81-186)), (S54-PADRE-sT(2-141)), (S54-PADRE-sT(81-141)) are expressed by the bacterial vector LT^{VAX} but none of them secrete and transfer outside of the bacterial vector despite the presence of the secretion peptide signal S54.

These experiments demonstrate that it is not easy to predict whether a fusion protein can be secreted and transferred, preferably translocated, by a bacterial vector owning a type 3 secretion system.

DCs that have Received LT (259, E216→K) Thanks to the Vector LT^{VAX} are Efficient for LT Presentation to T-Cells

LT^{VAX} delivers the fusion protein comprising the LT truncated form (2-259, E216→K) antigen tagged by the NP68 peptide. This peptide is frequently used in immunology research to sensitively trace the immune response. This is possible by using T-cells derived from F5 transgenic mice, which have high frequency of NP68-specific T-cells. This tool is particularly useful as there is no immunomonitoring tool specific for LT available on the market.

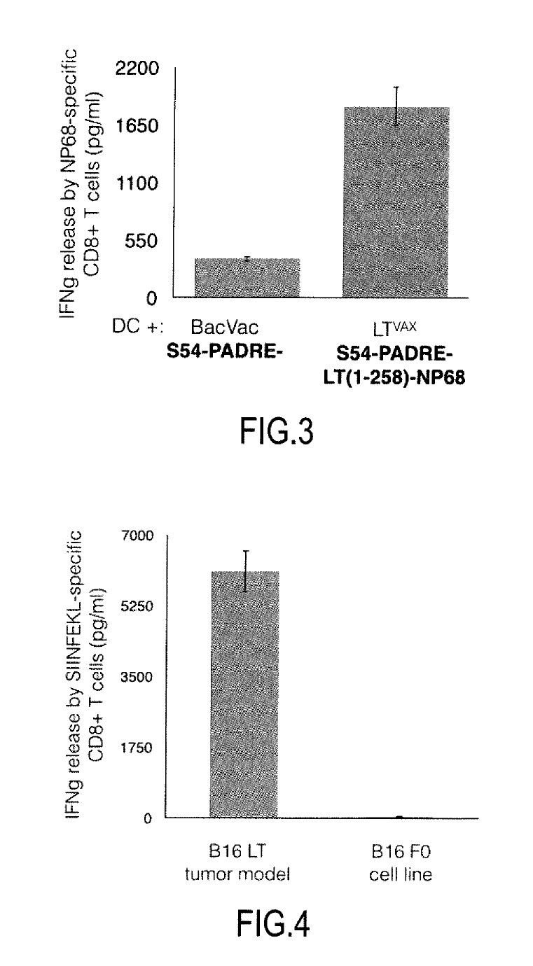

As shown in FIG. 3, IFNg release is significantly higher when activated NP68-specific T-cells were co-cultivated with DCs previously co-cultivated with LT^{VAX} compared to the negative control (NP68-specific T-cells co-cultivated with DCs previously co-cultivated with BacVac™). This reveals that the fusion protein comprising the LT truncated form (2-259, E216→K) as delivered by LT^{VAX} to DCs was efficiently processed for presentation to CD8 T-cells.

### B16 LT Tumor Cells are Efficient for LT Presentation to T-Cells

The LT-IRES-GFP plasmid used for the generation of the B16 LT tumor cells expresses an mRNA encoding two distinct proteins: (i) LT (2-259, E216→K) tagged by the SIINFEKL peptide and (ii) GFP. The plasmid also expresses the gene conferring resistance to geneticin. Following the isolation of cells that stably integrated the plasmid in the genome by prolonged treatment with geneticin, GFP-positive clones were isolated.

The SIINFEKL peptide was tagged to LT (2-259, E216→K) in order to confirm that LT (2-259, E216→K) is expressed and processed by the B16 LT tumor cells for presentation to T-cells. Consistently with this hypothesis, IFNg was released by activated, SIINFEKL-specific CD8 T-cells, when co-incubated with B16 LT tumor cells (FIG. 4). Instead, no IFNg was detected when the same T-cells were co-incubated with the parental tumor cell line (B16F0). This demonstrates that the B16 LT tumor cells properly express LT (2-259, E216→K) and processed it for presentation to CD8 T-cells.

### LT^{VAX} is Efficient for Tumor Growth Control

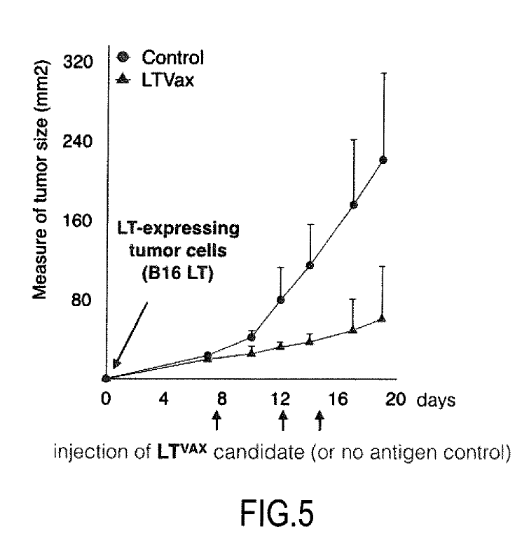

The efficacy of LT^{VAX} was demonstrated in an in vivo tumor challenge experiment: B16 LT tumor cells were implanted in mice, and LT^{VAX} treatment was performed when the tumor became palpable, and then repeated every three-four days throughout the experiment. LT^{VAX} significantly reduced the tumoral growth (p<0.5, T test), with a reduction of the tumor size of approximately 75% relative to tumor-bearing mice treated with the BacVac™ control vector (delivering no antigen), as measured 19 days post tumor implantation (FIG. 5).

### BIBLIOGRAPHY

1. Forbes N S, Nat. Rev. Cancer (2010) 10: 785 Engineering the perfect (bacterial) cancer therapy.
2. Bachmann M F & Jennings G T, Nat. Rev. Immunol. (2010) 10: 787 Vaccine delivery: a matter of size, geometry, kinetics and molecular patterns.
3. Banchereau J & Palucka A K, Nat. Rev. Immunol. (2005) 5: 296 Dendritic cells as therapeutic vaccines against cancer.
4. Radics et al, Nature Structural & Mol. Biol. (2014) 21 (1): 82 Structure of a pathogenic type 3 secretion system in action.
5. Chamekh M, Immunopharmacol. Immunotoxicol. (2010) 32: 1. Immunomodulation using genetically engineered bacteria for type III-mediated delivery of heterologous antigens and cytokines: potential application in vaccine and therapeutical developments.
6. Tseng et al, BMC Biotech. (2009) 9 (suppl 1): 52 Protein secretion systems in bacterial-host associations.
7. Hegazy W A et al, Infect Immun. (2012) 80: 1193 Evaluation of Salmonella enterica type III secretion system effector proteins as carriers for heterologous vaccine antigens.
8. Spreng S et al, Methods (2006) 38: 133 Rational design of Salmonella-based vaccination strategies.
9. Stocker B A. Vaccine (1988) 6: 141 Auxotrophic Salmonella typhi as live vaccine.
10. VanCott J L et al, Nat. Med. (1998) 4: 1247 Regulation of host immune responses by modification of Salmonella virulence genes.
11. Shuda et al, Proc. Nat. Acad. Sc. (2008) 105 (42): 16272 T antigen mutations are a human tumor-specific signature for Merkel cell polyomavirus.
12. Feng et al, Science (2008) 319 (5866): 1096 Clonal integration of a polyomavirus in human Merkel cell carcinoma.
13. Rodig et al, J Clin Invest. (2012) 122 (12): 4645 Improved detection suggests all Merkel cell carcinomas harbor Merkel polyomavirus.
14. Cheng et al, J. Virol. (June 2013) 87 (11): 6118 Merkel cell polyoma large T antigen has growth-promoting and inhibitory activities.
15. Zeng et al, Vaccine (2012) 30 (7): 1322 Development of a DNA vaccine targeting Merkel cell polyomavirus.
16. Gomez et al, Cell & Bioscience (2012) 2: 36 Strategy for eliciting antigen-specific CD8+ T-cell mediated immune response against a cryptic CTL epitope of Merkel cell polyoma virus large T antigen.
17. Basu et al, J. Exp. Med. (1999) 189 (5): 797
18. UJF WO 2005/049644, EP 1 692 162 and U.S. Pat. No. 7,939,319
19. UJF WO 2013/087667
20. Toussaint et al, Biochem. Biophys. Res. Commun. (1993) 196: 416
21. Polack B et al, Biochem. Biophys. Res. Commun. (2000) 275: 854 Protein delivery by Pseudomonas type III secretion system: Ex vivo complementation of p67(phox)-deficient chronic granulomatous disease.
22. Epaulard O et al, Mol. Ther. (2006) 14: 656 Anti-tumor immunotherapy via antigen delivery from a live attenuated genetically engineered Pseudomonas aeruginosa type Ill secretion system-based vector.
23. Epaulard O et al, Clin. Vaccine Immunol. (2008) 15: 308 Optimization of a type III secretion system-based Pseudomonas aeruginosa live vector for antigen delivery.
24. Le Gouëllec et al, Human vaccines & Immunotherapeutics (2012) 8 (10): 1454.
25. Le Gouëllec et al, Mol. Therapy (2013) 21 (5): 1076
26. Quénée et al, BioTechniques (2005) 38 (1): 63
27. Wang Y et al, J. Immunother. (2012) 35: 223 Optimization of antitumor immunotherapy mediated by type Ill secretion system-based live attenuated bacterial vectors.
28. Dubensky T W et al, Curr Opin Biotechnol. (2012) Killed but metabolically active vaccines.
29. Irsch J et al, Transfus. Med. Hemother. (2011) 38: 19 Inactivation of Platelet and Plasma Blood Components for Transfusion Using the INTERCEPT Blood System™.
30. Brockstedt D G et al, Nat. Med. (2005) 11: 853 Killed but metabolically active microbes: a new vaccine paradigm for eliciting effector T-cell responses and protective immunity.
31. Spreng S & Viret J F, Vaccine (2005) 23: 2060 Plasmid maintenance systems suitable for GMO-based bacterial vaccines.
32. Curtiss R, 3rd et al, Res. Microbiol. (1990) 141: 797 Stabilization of recombinant avirulent vaccine strains in vivo.
33. Andres et al, J. Cutan. Pathol. (2010) 37: 28
34. Cimino et al, Exp. & Mol. Pathol. (2013) 94: 40
35. DuThanh et al, J. Dermatol. Sc; (2013) 71: 138
36. Dworkin et al, J. Investigative Dermatol. (2009) 129: 2868
37. Imajoh et al, Virology (2012) 9: 154
38. Kassen et al, Int. J. Cancer (2009) 125: 356
39. Kreuter et al, Arch. Dermatol. (2011) 147 (12) 1449
40. Murakami et al, J Clin. Virol. (2011) 50: 37
41. Pantulu et al, Blood (2010) 116 (24): 5280
42. Scola et al, British J. Dermatology (2012) 167: 1315
43. Hashida et al, Br. J. Cancer. (2013 Feb. 19)108 (3): 629 Detection of Merkel cell polyomavirus with a tumour-specific signature in non-small cell lung cancer.
44. Antoniu et al, (2013) Int. J. Cancer (2013 Dec. 15)133 (12): 3016 Molecular pathological findings of Merkel cell polyomavirus in lung cancer: a possible etiopathogenetic link?
45. Karla et al, (2013) J. Am. Acad. Dermatol. (2013 June) 68 (6): 957 Cutaneous squamous cell carcinoma: estimated incidence of disease, nodal metastasis, and deaths from disease in the United States, 2012.
46. Mamalaki et al, Dev. Immunol. (1993) 3 (3): 159 Positive and negative selection in transgenic mice expressing a T-cell receptor specific for influenza nucleoprotein and endogenous superantigen.
47. Hogquist et al, Cell (1994) 76 (1): 17 T-cell receptor antagonist peptides induce positive selection.
48. Clarke et al, Immunol. Cell Biol. (2000) 78: 110 Characterization of the ovalbumin-specific TCR transgenic line OT-I: MHC elements for positive and negative selection.
49. Xu et al, Lung Cancer (2014) 83: 341
50. Megan et al, Virology (2013) 435 (1): 118 Merkel cell polyoma virus: A newly discovered human virus with oncogenic potential.
51. Showalter et al, Cell Host Microbe (2010) 7 (6): 509 Merkel cell polyoma virus and two polyoma viruses are chronically shed from human skin.
52. D'Addario et al, Annals of Oncology (2010) 21 (Suppl 5): 116
53. Lasithiotaki et al, Internat. J. Cancer (2013) 133: 604
54. DHHS Pittsburgh (Moore) WO 2009/079481 and U.S. Pat. No. 8,524,248
55. U. Florida WO 2012/012605 and U.S. Pat. No. 8,617,888
56. Institut Curie US 2011 182901
57. Alexander et al, Immunity (1994) 1 (9): 751
58. Needleman et al, J. Mol. Biol. (1970) 48: 444
59. Sambrook et al., 2012, Molecular Cloning: A Laboratory Manual, Fourth Edition, Cold Spring Harbor Laboratory Press.
60. H. A Carleton et al. Nature communication Nature Communications 4, Article number: 1590 doi:10.1038/ncomms2594
61. Edgren T, Forsberg A, Rosqvist R, Wolf-Watz H. PLoS Pathog. 2012; 8(5):e1002669. doi: 10.1371/journal.ppat.1002669. Epub 2012 May 10. Type III secretion in Yersinia: injectisome or not?
62. Le Gouëllec A, Chauchet X, Laurin D, Aspord C, Verove J, Wang Y, Genestet C, Trocme C, Ahmadi M, Martin S, Broisat A, Cretin F, Ghezzi C, Polack B, Plumas J, Toussaint B.
Mol Ther. 2013 May; 21(5):1076-86. doi: 10.1038/mt.2013.41. Epub 2013 Mar. 26.
PMID: 23531551
A safe bacterial microsyringe for in vivo antigen delivery and immunotherapy.
63. Krall R1, Zhang Y, Barbieri J T. J Biol Chem. 2004 Jan. 23; 279(4):2747-53. Epub 2003 Nov. 3.
Intracellular membrane localization of pseudomonas ExoS and Yersinia YopE in mammalian cells.

1. A fusion protein which comprises from its N-terminal end to its C-terminal end and fused in frame:
at least one secretion peptide signal able to direct said fusion protein to the type 3 secretion system of a bacterial vector, when said fusion protein is in a bacterial vector owning a type 3 secretion system, and
one truncated form of the Large T (LT) antigen of the Merkel Cell Polyoma Virus (MCPyV), which has an amino acid sequence having at least 80% identity with one of the amino acid sequences shown in SEQ ID NO: 1 which starts with the amino acid in any one of positions 1 to 5 and ends with the amino acid in any one of the positions 210 to 469.

2. The fusion protein according to claim 1, wherein the truncated form of the Large T antigen of the Merkel Cell Polyoma Virus has:
an amino acid sequence having at least 80% identity with the amino acid sequence shown in SEQ ID NO: 1 which starts with the amino acid in position 2 and ends with the amino acid in position 215 and provided that said truncated form of the Large T antigen of the Merkel Cell Polyoma Virus does not bind to the human retinoblastoma protein, or
an amino acid sequence having at least 80% identity with the amino acid sequence shown in SEQ ID NO: 1 which starts with the amino acid in position 2 and ends with the amino acid in position 270, and provided that said truncated form of the Large T antigen of the Merkel Cell Polyoma Virus does not bind to the human retinoblastoma protein, or
one of the amino acid sequences shown in SEQ ID NO: 1 which starts with the amino acid in any one of positions 1 to 5, advantageously in position 2, and ends with the amino acid in any one of positions 210 to 215, so that said truncated form of the Large T antigen of the Merkel Cell Polyoma Virus does not bind to the retinoblastoma protein, or
one of the amino acid sequences shown in SEQ ID NO: 1 which starts with the amino acid in any one of positions 1 to 5, advantageously in position 2, and ends with the amino acid in any one of positions 216 to 270, which is further mutated in the human retinoblastoma protein binding site located from positions 212 to 216 in SEQ ID NO: 1, so that said truncated form of the Large T antigen of the Merkel Cell Polyoma Virus does not bind to the human retinoblastoma protein.

3. The fusion protein according to claim 2, wherein the truncated form of the Large T antigen of the Merkel Cell Polyoma Virus has one of amino acid sequences shown in SEQ ID NO: 1 which starts with the amino acid in any one of positions 1 to 5, advantageously in position 2, and ends with the amino acid in position 215.

4. A fusion protein according to claim 2, wherein the truncated form of the Large T antigen of the Merkel Cell Polyoma Virus has:
one of the amino acid sequences shown in SEQ ID NO: 1 which starts with the amino acid in any one of positions 1 to 5, advantageously in position 2, and ends with the amino acid in any one of positions 250 to 260, advantageously in position 259, which is further mutated in the human retinoblastoma protein binding site located from positions 212 to 216 in SEQ ID NO: 1, so that said truncated form of the Large T antigen of the Merkel Cell Polyoma Virus does not bind to the human retinoblastoma protein; or
an amino acid sequence having at least 85% identity with the amino acid sequence shown in SEQ ID NO: 1 which starts with the amino acid in position 2 and ends with the amino acid in position 259 and provided that said truncated form of the Large T antigen of the Merkel Cell Polyoma Virus does not bind to the human retinoblastoma protein.

5. The fusion protein according to claim 2, wherein the fusion protein is mutated in the human retinoblastoma protein binding site in position 216 in SEQ ID NO: 1.

6. The fusion protein according to claim 5, wherein the mutation in position 216 in SEQ ID NO: 1 is a substitution mutation replacing the Glu residue by a Lys residue (E216→K).

7. The fusion protein according to claim 1, wherein the secretion peptide signal that is able to direct said fusion protein to the type 3 secretion system of a bacterial vector is the N-terminal moiety of the Pseudomonas exoS gene product and has one of the amino acid sequences shown in SEQ ID NO: 2 which starts with the amino acid in position 1 and ends with the amino acid in any one of positions 15 to 129, advantageously ends with the amino acid in any one of positions 15 to 70, advantageously ends with the amino acid in position 54.

8. The fusion protein according to claim 1, wherein it further comprises a Pan-HLA-DR-binding epitope.

9. A bacterial vector owning a type 3 secretion system which is able to express, secrete and transfer, preferably translocate, into mammalian cells, the fusion protein as defined in claim 1.

10. The bacterial vector according to claim 9, wherein said bacterial vector is a bacterium, and in particular an attenuated bacterium.

11. The bacterial vector according to the claim 10, wherein the said bacterium comprises an expression cassette encoding a fusion protein inserted into the chromosome of the bacterium or inserted into a plasmid, the fusion protein comprising from its N-terminal end to its C-terminal end and fused in frame:
at least one secretion peptide signal able to direct said fusion protein to the type 3 secretion system of a bacterial vector, when said fusion protein is in a bacterial vector owning a type 3 secretion system, and
one truncated form of the Large T (LT) antigen of the Merkel Cell Polyoma Virus (MCPyV), which has an amino acid sequence having at least 80% identity with one of the amino acid sequences shown in SEQ ID NO: 1 which starts with the amino acid in any one of positions 1 to 5 and ends with the amino acid in any one of the positions 210 to 469.

12. The bacterial vector according to claim 10, wherein the bacterium belongs to the genus of Pseudomonas, in particular wherein the bacterium belongs to the Pseudomonas aeruginosa species or to the Pseudomonas syringuae species.

13. The bacterial vector according to claim 10, which is unable to express at least one of the products chosen among the exoS, exoT, exoU and exoY gene products and NDK cytotoxin, preferably which is unable to express at least the exoS, exoT and exoU gene products.

14. The bacterial vector according to any one of the claims 10 to 13, which comprises an expression cassette, wherein the nucleotide sequence encoding a fusion protein comprising from its N-terminal end to its C-terminal end and fused in frame:
at least one secretion peptide signal able to direct said fusion protein to the type 3 secretion system of a bacterial vector, when said fusion protein is in a bacterial vector owning a type 3 secretion system, and
one truncated form of the Large T (LT) antigen of the Merkel Cell Polyoma Virus one truncated form of the large T (LT) antigen of the Merkel Cell Polyoma Virus (MCPyV), which has an amino acid sequence having at least 80% identity with one of the amino acid sequences shown in SEQ ID NO: 1 which starts with the amino acid in any one of positions 1 to 5 and ends with the amino acid in any one of the positions 210 to 469 is placed under the control of the exoS promoter.

15. The bacterial vector according to claim 9, for use in a method of preventing or combating MCPyV infection, in particular by promoting a CD8+ immune response against MCPyV-infected cells.

16. The bacterial vector for use according to claim 15, wherein the CD8+ immune response is cytotoxic.

17. The bacterial vector for use according to claim 15, wherein the immune response is against cells (a) expressing whole or part of the MCPyV LT antigen and (b) in the genome of which is integrated an MCPyV nucleotide sequence encoding said whole or part of the LT antigen.

18. The bacterial vector for use according to claim 15, wherein the method of combating or preventing MCPyV infection prevents the onset of a tumoral disorder in patients infected with MCPyV.

19. The bacterial vector for use according to claim 15, wherein the method of combating or preventing MCPyV infection reduces the growth or propagation of a tumor in a patient suffering from a tumoral disorder characterized by the presence of tumoral cells expressing the LT antigen of MCPyV.

20. The bacterial vector according to claim 9, for use as a medicament, preferably for use as a medicament in the treatment of cancer.
